(19) United States
(12) Patent Application Publication
(10) Pub. No.: US 2024/0412808 A1
(43) Pub. Date: Dec. 12, 2024

(54) DETECTION OF CYSTIC FIBROSIS TRANSMEMBRANE CONDUCTANCE REGULATOR POLYTG/POLYT VARIATIONS BY AN NGS-BASED METHOD

(71) Applicant: Laboratory Corporation of America Holdings, Burlington, NC (US)

(72) Inventors: Qiandong Zeng, Belmont, MA (US); Yuanyu Cao, Carlsbad, CA (US)

(21) Appl. No.: 18/739,059

(22) Filed: Jun. 10, 2024

Related U.S. Application Data
(60) Provisional application No. 63/507,044, filed on Jun. 8, 2023.

(51) Int. Cl. G16B 5/00; G16B 30/10

(52) U.S. Cl. CPC G16B 5/00; G16B 30/10

(57) ABSTRACT

The present disclosure relates to a computational strategy with a digital read-out for detecting variants in a targeted genomic region. Particularly, aspects are directed to generating simulated variants for the targeted genomic region, detecting zero or more variations in a first allele, and zero or more variations in a second allele by inputting the simulated sequencing files into a sequencing analysis pipeline for alignment to a reference genome, identifying the zero or more variations between the first and second allele's simulated sequencing read file and the reference genome, and outputting a variant table comprising the zero or more detected variants and their corresponding variant features, concatenating the variant features for the zero or more detected variants in the first allele and in the second allele to generate a sequencing fingerprint profile, and outputting, the sets of simulated variants and their corresponding sequencing fingerprint profiles into a truth fingerprint table.

### CROSS REFERENCE TO RELATED APPLICATION

The present application claims priority and benefit from U.S. Provisional Patent Application No. 63/507,044, filed Jun. 8, 2023, the entire contents of which are incorporated herein by reference for all purposes.

### FIELD

The present disclosure relates to cystic fibrosis genetic diagnosis, and in particular to a computational strategy with a digital read-out for detecting variants in the cystic fibrosis transmembrane conductance regulator (CFTR) polyTG/polyT region.

### BACKGROUND

Next generation sequencing (NGS) is increasingly being used in clinical genetic testing to aid in diagnosis and patient care, providing highly sensitive and accurate high-throughput platforms for large-scale genomic testing. Whole-genome sequencing (WGS) is a comprehensive NGS method for analyzing entire genomes (sequences all or a substantially all 3 billion DNA base pairs that make up an entire genome by determining the order of the nucleotides (A, C, G, T)). The goal of whole-genome sequencing is, typically, to look for genetic aberrations (e.g., single nucleotide variants, deletions, insertions and copy number variants). Because the entire genome is being sequenced, changes in the noncoding sections of DNA within genes, called introns, can also be determined.

An alternative approach to WGS is to only sequence the protein-coding regions called exons, using whole-exome sequencing (WES). Exons compose only about 2% of the whole human genome, and thus can be sequenced at a much greater depth (number of times a given nucleotide is sequenced) for lower cost. This greater depth provides more confidence in the detection of low frequency alterations. Another, more specific sequencing strategy is targeted genomic sequencing (TGS), which focuses on a panel of target genes or genomic regions known to have strong associations with pathogenesis of disease and/or clinical relevance. Accordingly, these targeted regions are covered with even greater sequencing depth with reduced costs and data burden. This allows targeted sequencing to identify low frequency variants in targeted regions with high confidence, thus suitable for profiling low-quality and fragmented clinical DNA samples.

### BRIEF SUMMARY

In various embodiments, a computer implemented method comprises: generating, using a NGS simulation tool, sets of simulated variants and corresponding simulated sequencing read files for a targeted genomic region, wherein: each of the sets of simulated variants from the sets of simulated variants comprise a first allele with zero or more mutations, and a second allele with zero or more mutations, and the simulated sequencing read files comprise (i) a forward sequencing read file comprising at least 10,000 simulated reads, (ii) a reverse sequencing read file comprising at least 10,000 simulated reads, or both (i) and (ii); detecting, using the simulated sequencing files, zero or more variations in the first allele, and zero or more variations in the second allele, wherein detecting comprises: inputting the simulated sequencing files into a sequencing analysis pipeline for alignment to a reference genome, identifying the zero or more variations between the first allele's simulated sequencing read file and the reference genome and the zero or more variations between the second allele's simulated sequencing read file and the reference genome, and outputting a variant table comprising the zero or more detected variants and their corresponding variant features; concatenating the variant features for the zero or more detected variants in the first allele and the variant features for the zero or more detected variants in the second allele to generate a sequencing fingerprint profile; and outputting, into a truth fingerprint table, the sets of simulated variants and their corresponding sequencing fingerprint profiles.

In some embodiments, the targeted genomic region is ±500 base pairs of the CFTR poly TG/polyT region.

In some embodiments, the first allele and the second allele comprise the same mutations, different mutations, or any combination thereof.

In some embodiments, the sequencing fingerprint profile comprises the starting coordinate for the variant, the reference genome nucleic acid base, the alternative nucleic acid base, the variant type, the zygosity of the variant, or any combination thereof.

In some embodiments, the outputting further comprises deduplicating the sequencing fingerprint profile to remove simulated variants from the truth fingerprint table that generate the same sequencing fingerprint profile.

In various embodiments, a computer implemented method comprises: performing a next generation sequencing assay on a sample, wherein the NGS assay generates sample sequencing read files for the targeted genomic region, wherein the sample sequencing read files comprise (i) a forward sequencing read file, (ii) a reverse sequencing read file, or both (i) and (ii); processing, using a sequencing analysis pipeline, the sample sequencing read files for variant detection, wherein processing comprises: inputting the sample sequencing read files into a sequencing analysis pipeline for alignment to a reference genome, identifying zero or more variations between the sequencing read files and the reference genome, and outputting a variant table comprising the zero or more detected variants and their corresponding variant features; concatenating the variant features for the zero or more detected variants to generate a sample fingerprint profile; accessing a truth fingerprint table comprising sets of simulated variants and their corresponding sequencing fingerprint profiles; cross-referencing the sample fingerprint profile with the sequencing fingerprint profiles in the truth fingerprint table to determine whether a match exists; when a match exists between the sample fingerprint profile and the sequencing fingerprint profiles in the truth fingerprint table, (i) generating a report comprising the sample fingerprint profile and the detected variants in the sample; and when a match does not exist between the sample fingerprint profile and the sequencing fingerprint profiles in the truth fingerprint table, (i) updating the truth fingerprint table to include the sample fingerprint profile, (ii) repeating the cross-referencing for the sample fingerprint table, and (iii) generating a report comprising the sample fingerprint profile and the detected variants in the sample.

In some embodiments, the sample is a cell-containing liquid or a tissue comprising nucleic acid molecules.

In some embodiments, the sample is a blood sample.

In some embodiments, the NGS assay is a targeted NGS assay for a targeted genomic region.

In some embodiments, the targeted genomic region is ±500 base pairs of the CFTR polyTG/polyT region.

In some embodiments, the method further comprises when the sequencing analysis pipeline is unable to make a definitive variant call from the sample sequencing read files: accessing, using the sequencing analysis pipeline, the sample sequencing read files to count the number of reads that align to the detected variant; and resolving, based on the sequencing read counts, the fingerprint profile for the detected variant.

In various embodiments, a system comprises: one or more processors; and one or more computer-readable media storing instructions which, when executed by the one or more processors, cause the system to perform operations comprising: generating, using a NGS simulation tool, at least 5,000 sets of simulated variants and corresponding simulated sequencing read files for a targeted tandem repeat genomic region, wherein: each of the sets of simulated variants from the at least 5,000 sets of simulated variants comprise a first allele with zero or more mutations, and a second allele with zero or more mutations, and the simulated sequencing read files comprise (i) a forward sequencing read file comprising at least 10,000 simulated reads, (ii) a reverse sequencing read file comprising at least 10,000 simulated reads, or both (i) and (ii); detecting, using the simulated sequencing files, zero or more variations in the first allele and zero or more variations in the second allele, wherein detecting comprises: inputting the simulated sequencing files into a sequencing analysis pipeline for alignment to a reference genome, identifying the zero or more variations between the first allele's simulated sequencing read file and the reference genome and the zero or more variations between the second allele's simulated sequencing read file and the reference genome, and outputting a variant table comprising the zero or more detected variants and their corresponding variant features; concatenating the variant features for the zero or more detected variants in the first allele and the variant features for the zero or more detected variants in the second allele to generate a sequencing fingerprint profile; outputting, into a truth fingerprint table, the at least 5,000 sets of simulated variants and their corresponding sequencing fingerprint profiles; performing an NGS assay on a sample, wherein the NGS assay generates sample sequencing read files for the targeted tandem repeat genomic region, wherein the sample sequencing read files comprise (i) a forward sequencing read file, (ii) a reverse sequencing read file, or both (i) and (ii); processing, using a sequencing analysis pipeline, the sample sequencing read files for variant detection, wherein processing comprises: inputting the sample sequencing read files into a sequencing analysis pipeline for alignment to a reference genome, identifying zero or more variations between the sample sequencing read files and the reference genome, and outputting a variant table comprising the zero or more detected variants and their corresponding variant features; concatenating the variant features for the zero or more detected variants to generate a sample fingerprint profile; cross-referencing the sample fingerprint profile with the sequencing fingerprint profiles in the truth fingerprint table to determine whether a match exists; when a match exists between the sample fingerprint profile and the sequencing fingerprint profiles in the truth fingerprint table, (i) generating a report comprising the sample fingerprint profile and the detected variants in the sample; and when a match does not exist between the sample fingerprint profile and the sequencing fingerprint profiles in the truth fingerprint table, (i) updating the truth fingerprint table to include the sample fingerprint profile, (ii) repeating the cross-referencing for the sample fingerprint table, and (iii) generating a report comprising the sample fingerprint profile and the detected variants in the sample.

In some embodiments, the tandem repeat genomic region is ±500 base pairs of the CFTR polyTG/polyT region.

In some embodiments, the first allele and the second allele comprise the same mutations, different mutations, or any combination thereof.

In some embodiments, the sequencing fingerprint profile comprises the starting coordinate for the variant, the reference genome nucleic acid base, the alternative nucleic acid base, the variant type, the zygosity of the variant, or any combination thereof.

In some embodiments, the outputting further comprises deduplicating the sequencing fingerprint profile to remove simulated variants from the truth fingerprint table that generate the same sequencing fingerprint profile.

In some embodiments, the sample is a cell-containing liquid or a tissue comprising nucleic acid molecules, and wherein the sample is a blood sample.

In some embodiments, the NGS assay is a targeted NGS assay for a tandem repeat genomic region, and wherein the tandem repeat genomic region is ±500 base pairs of the CFTR polyTG/polyT region.

In some embodiments, the sample fingerprint profile comprises the starting coordinate for the variant, the reference genome nucleic acid base, the alternative nucleic acid base, the variant type, the zygosity of the variant, or any combination thereof.

In some embodiments, the operations further comprise: when the sequencing analysis pipeline is unable to make an accurate variant call from the sample sequencing read files: accessing, using the sequencing analysis pipeline, the sample sequencing read files to count the number of reads that align to the detected variant; and resolving, based on the sequencing read counts, the fingerprint profile for the detected variant.

In some embodiments, a system is provided that includes one or more data processors and a non-transitory computer readable medium containing instructions which, when executed on the one or more data processors, cause the one or more data processors to perform part or all of one or more methods or processes disclosed herein.

In some embodiments, a computer-program product is provided that is tangibly embodied in a non-transitory machine-readable medium and that includes instructions configured to cause one or more data processors to perform part or all of one or more methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The patent or application file contains at least one drawing executed in color. Copies of this patent or patent application with color drawing(s) will be provided by the Office up request and payment of the necessary fee.

Aspects and features of the various embodiments will be more apparent by describing examples with reference to the accompanying drawings, in which:

FIG. 1 shows an illustration of NGS read alignment in the CFTR polyTG and polyT region on human chromosome 7.

FIG. 2 shows a computing environment in accordance with various embodiments.

FIG. 3 shows a flowchart illustrating a method for generating sequencing fingerprint profiles in accordance with various embodiments.

FIG. 4 shows a flowchart illustrating deployment of a truth fingerprint table in a clinical setting in accordance with various embodiments.

FIG. 5 shows a block diagram illustrating a computing environment in which various systems, methods, algorithms, machine-learning models, and data structures may be implemented in accordance with various embodiments.

FIG. 6 illustrates an exemplary computational strategy with a digital read-out for detecting NGS variants in the CFTR polyTG/polyT region in accordance with various embodiments.

FIG. 7 shows a comparison between Sanger sequencing results (FIG. 7A) and NGS sequencing results (FIG. 7B) in accordance with various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### TERMS

Articles “a” and “an” are used herein to refer to one or to more than one (i.e. at least one) of the grammatical object of the article. By way of example, “an element” means at least one element and can include more than one element.

The use of any and all examples or exemplary language (e.g., “such as”) provided herein, is intended merely to better illustrate the invention and does not pose a limitation on the scope of the invention unless otherwise claimed.

The terms “may,” “may be,” “can,” and “can be,” and related terms are intended to convey that the subject matter involved is optional (that is, the subject matter is present in some examples and is not present in other examples), not a reference to a capability of the subject matter or to a probability, unless the context clearly indicates otherwise.

As used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, and 10 percent. As used herein, when an action is “based on” something, this means the action is based at least in part on at least a part of the something.

The use herein of the terms “including,” “comprising,” or “having” and variations thereof, is meant to encompass the elements listed thereafter and equivalents thereof as well as additional elements. Embodiments recited as “including,” “comprising,” or “having” certain elements are also contemplated as “consisting essentially of” and “consisting of” those certain elements. As used herein, “and/or” refers to and encompasses any and all possible combinations of one or more of the associated listed items, as well as the lack of combinations were interpreted in the alternative (“or”).

Recitation of ranges of values herein are merely intended to serve as a shorthand method of referring individually to each separate value falling within the range, unless otherwise indicated herein, and each separate value is incorporated into the specification as if it were individually recited herein. For example, if a range is stated as between 50-300, it is intended that values such as 51, 52, 53, 54, 55, 60, 65, 70, 80, 90, 100, 150, 200, 300 etc., are expressly enumerated in this specification. These are only examples of what is specifically intended, and all possible combinations of numerical values (e.g., integer, whole number, decimal, fraction, and the like) between and including the lowest value and the highest value enumerated are to be considered to be expressly stated in this disclosure.

As used throughout, the term “nucleic acid” or “nucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar properties as the reference nucleic acid and are metabolized in a manner similar to naturally occurring nucleotides. A nucleic acid sequence can comprise combinations of deoxyribonucleic acids and ribonucleic acids. Such deoxyribonucleic acids and ribonucleic acids include both naturally occurring molecules and synthetic analogues. The polynucleotides of the invention also encompass all forms of sequences including, but not limited to, single-stranded forms, double-stranded forms, hairpins, stem-and-loop structures, and the like.

In general, the term “sequence variant” refers to any variation in sequence relative to one or more reference sequences. Typically, the sequence variant occurs with a lower frequency than the reference sequence for a given population of individuals for whom the reference sequence is known. In some cases, the reference sequence is a single known reference sequence, such as the genomic sequence of a single individual. In some cases, the reference sequence is a consensus sequence formed by aligning multiple known sequences, such as the genomic sequence of multiple individuals serving as a reference population, or multiple sequencing reads of polynucleotides from the same individual. In some cases, the sequence variant occurs with a low frequency in the population (also referred to as a “rare” sequence variant). For example, in non-tissue samples, the sequence variant may occur with a frequency of about or less than about 5%, 4%, 3%, 2%, 1.5%, 1%, 0.75%, 0.5%, 0.25%, 0.1%, 0.075%, 0.05%, 0.04%, 0.03%, 0.02%, 0.01%, 0.005%, 0.001%, or lower. A sequence variant can be any sequence that varies from a reference sequence. A sequence variation may consist of a change in, insertion of, or deletion of a single nucleotide, or of a plurality of nucleotides (e.g., 2, 3, 4, 5, 6, 7, 8, 9, 10, or more nucleotides). Where a sequence variant includes two or more nucleotide differences, the nucleotides that are different may be contiguous with one another, or discontinuous. Non-limiting examples of types of sequence variants include single nucleotide polymorphisms (SNP), deletion/insertion polymorphisms (INDEL), copy number variants (CNV), loss of heterozygosity (LOH), microsatellite instability (MSI), variable number of tandem repeats (VNTR), and retrotransposon-based insertion polymorphisms. Additional examples of types of sequence variants include those that occur within short tandem repeats (STR) and simple sequence repeats (SSR), or those occurring due to amplified fragment length polymorphisms (AFLP). In some aspects, a sequence variant can refer to a chromosome rearrangement, including but not limited to a translocation or fusion gene, or rearrangement of multiple genes resulting from, for example, chromothripsis.

Unless defined otherwise, all technical and scientific terms used herein have the same meaning as commonly understood by one of ordinary skill in the art to which this invention belongs. Although any methods and materials similar to or equivalent to those described herein can be used in the practice or testing of the invention, the preferred methods and materials are now described.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function.

### I. Introduction

Cystic fibrosis (CF) is a lifelong debilitating disease that is typically diagnosed in children by 2 years of age. Characterized by abnormally viscous mucosal secretions in the airways of the lungs, pancreatic ducts, and other organ systems composed of epithelial tissue, individuals diagnosed with CF are at increased risk of developing infections and suffering from malnourishment. Advancements in treatment and patient care have greatly improved the quality of life of affected individuals; however, CF is still associated with a reduced life expectancy and high treatment burden related to its multisystem manifestations.

From the genetic perspective, CF is an autosomal recessive condition that primarily affects those of European descent (prevalence of 1 in ˜3000 live births). Autosomal recessive refers to when both inherited copies (e.g., alleles) of a gene are mutated resulting in a disease phenotype. Genetic mutations in the cystic fibrosis transmembrane conductance regulator (CFTR) gene are responsible for the classical symptoms observed in CF patients. CFTR functions as an anion channel membrane protein at the surface of cells and facilitates the movement of chloride and bicarbonate ions across epithelial cell membranes. Specifically, CFTR supports active fluid transport and movement homeostasis across epithelial layers. Mutations that cause a decrease or loss of protein function can cause aberrant ion and fluid homeostasis and the buildup of the thick mucus layer characteristic of CF. Despite being a monogenic disorder, more than 2,000 CFTR mutations have been described, wherein the most common mutation is a 3 base pair deletion of a single phenylalanine (F) residue at position 508 (AF508) in the protein. The AF508 deletion induces protein misfolding which ultimately results in failure of the protein to be translocated to the cell surface. Moreover, this deletion accounts for nearly 70% of all cystic fibrosis cases and is carried in 1.5% of the population. In addition to AF508, many of the other CFTR pathogenic mutations that have been linked to cystic fibrosis, are detectable via NGS with high sensitivity and specificity.

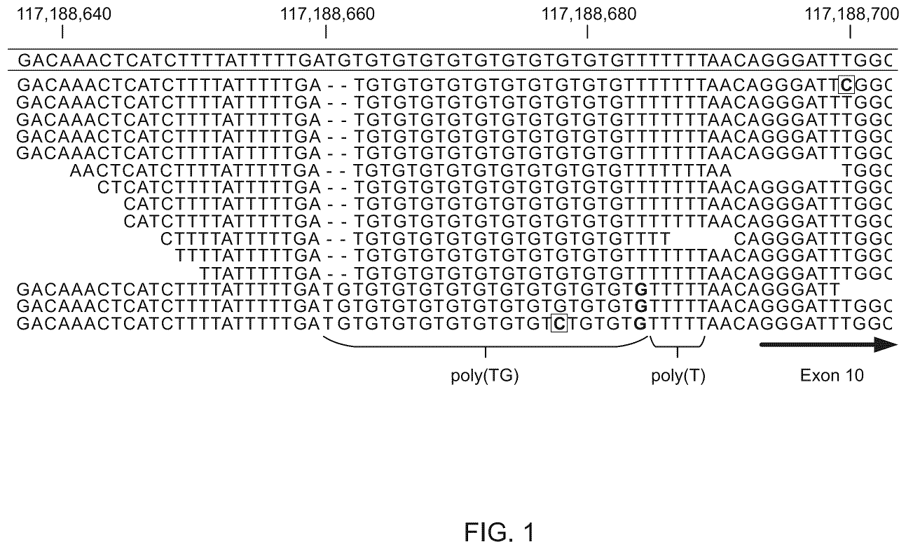

A critical region of the CFTR gene is the polyTG/polyT tract located immediately upstream of exon 10, which serves as the splice acceptor site for the inclusion of exon 9 during RNA splicing. In most mammalian genomes, polypyrimidine tracts (polyT or polyC tracts) at the splice acceptor cite are highly conserved, as they are necessary for RNA splicing. RNA splicing refers to the post-transcriptional process of removing introns and ligating exons together to generate a mature mRNA. Alterations to the polypyrimidine tract increase the risk of inappropriate inclusion or exclusion of exons and potentially result in a truncated, non-functional protein, as is the case with CFTR. Wild type CFTR encodes a string of 11 TG-repeats immediately followed by a string of 7 Ts, as shown in FIG. 1. The polyTG/polyT tract ensure the inclusion of exon 9 during conical RNA splicing. However, shorter polyT tracts (5 Ts or less) have been linked to inappropriate exclusion of exon 9, resulting in reduced levels of CFTR mRNA and protein expression, potentially leading to CFTR-related disorders.

PolyTG and polyT tracts are also examples of low complexity repeat regions within the genome, which are regions that contain simple sequence repeats. They are highly unstable and susceptible to replication slippage leading to uncontrolled expansion. When expansion occurs in protein coding genes, frequently the overall structure and function of the protein are affected, often manifesting in disease. Another concern of low complexity repeat regions is that they are notoriously difficult to obtain accurate alignments using NGS methods. Accordingly, Sanger sequencing is used as the gold standard for confirmation of NGS variants outside of regions with poor mappability (areas with homology, low complexity sequence/repeat elements). An additional concern for the polyTG/polyT tract in CFTR is that the lengths of the tracts can vary between the two alleles in a patient sample (e.g., haplotype), further complicating alignment and interpretation of NGS data. Because Sanger sequencing uses an analog signal, it is challenging for Sanger sequencing to disentangle haplotype alleles, especially in the CFTR polyTG/polyT tract region. Given that NGS is one of the most utilized diagnostic tools for CF, it is imperative that robust and reproducible workflows are available to accurately detect pathogenic or likely pathogenic variants in the polyTG/polyT tract of the CFTR gene.

To address these challenges and others, disclosed herein is a computational strategy for detecting variants in a targeted genomic location (also described herein as targeted genomic region) such as the polyTG/polyT tract of the CFTR gene. Specifically, simulated variant reads are generated for the targeted genomic location using a simulation tool. The simulated reads are input into a genomics analysis subsystem that generates an NGS variant table for each of the simulated input polyTG/polyT variants. The NGS variant table comprises features describing the output variant(s) detected by the NGS analysis pipeline. The features of the detected NGS variant table are concatenated to generate a mutation pattern (“fingerprint”) for the corresponding input poyTG/polyT variants. To account for cases where different input polyTG/polyT variants generate the same mutation pattern, deduplication techniques are used on the mutation patterns. The deduplicated mutation patterns are used to construct a mutation fingerprint lookup table comprising pairs of simulated variants with their corresponding mutation pattern. Clinical sample mutation profilers are cross-checked with the lookup table is used to compare variants found in clinical samples from the genomic location to provide accurate and highly confident polyTG/polyT allele calls.

The computational strategy disclosed above overcomes concerns regarding accurate NGS alignment and haplotype distinction by taking advantage of simulated variant reads and sequencing read data for the polyTG/polyT tract in CFTR to generate high-quality NGS-based fingerprint profiles for each simulated variant. The simulated variants comprise all possible combinations of polyT length ranging from T[1-13] and polyTG length ranging from TG[7-15]. Because the variants are simulated, the exact number of Ts and TGs are known for each allele. In other words, the simulation method generates high confident variant fingerprint profiles that are indicative of their corresponding polyTG/polyT variant set. Accordingly, each set of simulated variants (one for each allele) and its corresponding fingerprint profile are stored in a “lookup” table that is used when assessing the presence and type of variant in a clinical sample. The sequencing and variant calling results for the clinical sample are used to generate a patient specific fingerprint profile which is compared to the high confident fingerprint profile “lookup” table to make the polyTG/poly T variant call. The accuracy of this approach was confirmed by Sanger sequencing, and shown to be 100% concordant with Sanger, or even more accurate than Sanger results in a number of cases. Furthermore, by focusing on the analysis of the polyTG/polyT track genomic region, as opposed to WES or WGS, the total computational run time is significantly reduced to less than one minute, e.g., approximately 30 seconds, for a flow cell with 20-200 samples, greatly improving practical application in clinical diagnosis and treatment.

### II. Computer Environment

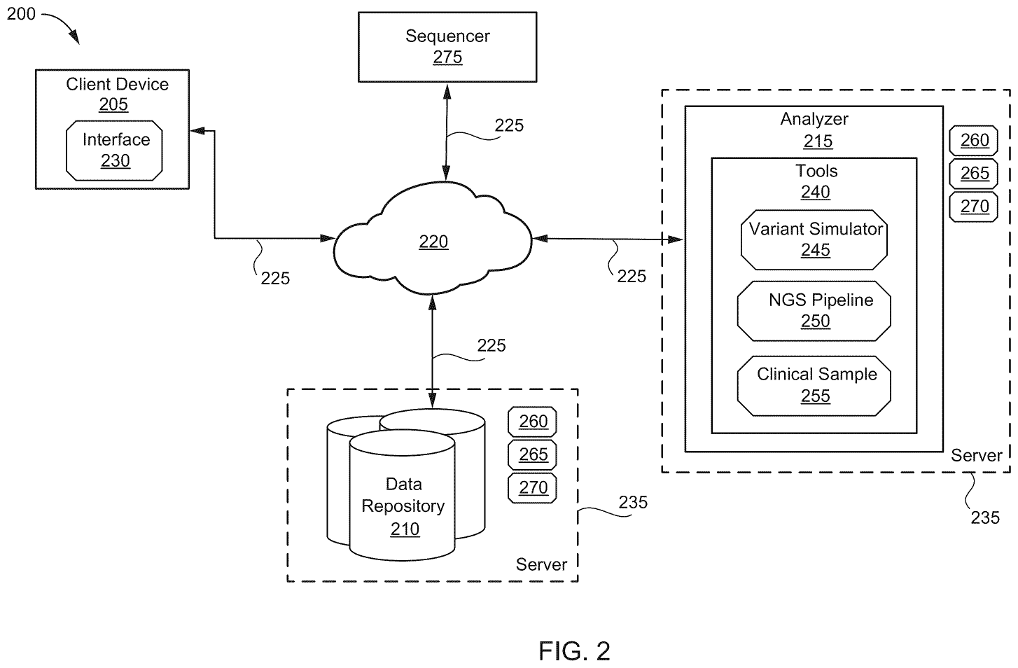

FIG. 2 shows a computing environment 200 in accordance with aspects of the present disclosure. Computing environment 200 includes a client device 205, a data repository 210, an analyzer 215, and a sequencer 275 connected to each other by a network 220. Although FIG. 2 illustrates a particular arrangement of a client device 205, a data repository 210, an analyzer 215, and a network 220, this disclosure contemplates any suitable arrangement of a client device 205, a data repository 210, an analyzer 215, a sequencer 275, and a network 220. As an example, and not by way of limitation, two or more client devices 205, a data repository 210, an analyzer 215, and a sequencer 275 may be connected to each other directly, bypassing network 220. As another example, two or more client devices 205, a data repository 210, an analyzer 215, and a sequencer 275 may be physically or logically co-located with each other in whole or in part. Moreover, although FIG. 2 illustrates a particular number of a client device 205, a data repository 210, an analyzer 215, a sequencer 275, and network 220, this disclosure contemplates any suitable number of client devices 205, data repositories 210, analyzers 215, a sequencer 275, and networks 220. As an example, and not by way of limitation, computing environment 200 may include multiple client devices 205, data repositories 210, analyzers 215, a sequencer 275, and network 220.

This disclosure contemplates any type of network 220 familiar to those skilled in the art that may support data communications using any of a variety of available protocols including without limitation TCP/IP (transmission control protocol/Internet protocol), SNA (systems network architecture), IPX (Internet packet exchange), AppleTalk®, and the like. Merely by way of example, network(s) 220 may be a local area network (LAN), networks based on Ethernet, Token-Ring, a wide-area network (WAN), the Internet, a virtual network, a virtual private network (VPN), an intranet, an extranet, a public switched telephone network (PSTN), an infra-red network, a wireless network (e.g., a network operating under any of the Institute of Electrical and Electronics (IEEE) 1002.11 suite of protocols, Bluetooth®, and/or any other wireless protocol), and/or any combination of these and/or other networks.

Links 225 may connect a client device 205, a data repository 210, and an analyzer 215 to a network 220 or to each other. This disclosure contemplates any suitable links 225. In particular embodiments, one or more links 225 include one or more wireline (such as for example Digital Subscriber Line (DSL) or Data Over Cable Service Interface Specification (DOCSIS)), wireless (such as for example Wi-Fi or Worldwide Interoperability for Microwave Access (WiMAX)), or optical (such as for example Synchronous Optical Network (SONET) or Synchronous Digital Hierarchy (SDH)) links. In particular embodiments, one or more links 225 each include an ad hoc network, an intranet, an extranet, a VPN, a LAN, a WLAN, a WAN, a WWAN, a MAN, a portion of the Internet, a portion of the PSTN, a cellular technology-based network, a satellite communications technology-based network, another link 225, or a combination of two or more such links 225. Links 225 need not necessarily be the same throughout a computing environment 200. One or more first links 225 may differ in one or more respects from one or more second links 225.

A client device 205 is an electronic device including hardware, software, or embedded logic components or a combination of two or more such components and capable of interacting with the data repository 210 and the analyzer 215 with respect to appropriate product target discovery functionalities in accordance with techniques of the disclosure. The client devices may include several types of computing systems such as portable handheld devices, general purpose computers such as personal computers and laptops, workstation computers, wearable devices, gaming systems, thin clients, various messaging devices, sensors, or other sensing devices, and the like. These computing devices may run various types and versions of software applications and operating systems (e.g., Microsoft Windows®, Apple Macintosh®, UNIX® or UNIX-like operating systems, Linux or Linux-like operating systems such as Google Chrome™ OS) including various mobile operating systems (e.g., Microsoft Windows Mobile®, iOS®, Windows Phone®, Android™, BlackBerry®, Palm OS®). Portable handheld devices may include cellular phones, smartphones, (e.g., an iPhone), tablets (e.g., iPad®), personal digital assistants (PDAs), and the like. Wearable devices may include Google Glass® head mounted display, and other devices. Client device 205 may be capable of executing various applications such as various Internet-related apps, communication applications (e.g., E-mail applications, short message service (SMS) applications) and may use various communication protocols. This disclosure contemplates any suitable client device 205 configured to generate and output product target discovery content to a user. For example, users may use client device 205 to execute one or more applications, which may generate one or more discovery or storage requests that may then be serviced in accordance with the teachings of this disclosure. A client device 205 may provide an interface 230 (e.g., a graphical user interface) that enables a user of the client device 205 to interact with the client device 205. Client device 205 may also output information to the user via this interface 230. Although FIG. 2 depicts only one client device 205, any number of client devices 205 may be supported.

A data repository 210 is a data storage entity (or sometimes entities) into which data has been specifically partitioned for an analytical or reporting purpose. The data repository 210 may be used to store data and other information for use by the analyzer 215 and client device 205. For example, one or more of the data repositories 210 (a) and 210 (b) may be used to store data and information to be used as input into the analyzer 215 for generating a mutation pattern (e.g., a fingerprint) for a patient. In some instances, the data and information relate to various sequencing and/or variant call files from clinical samples obtained from patients at risk of, diagnosed with, or being treated for cystic fibrosis. The data repositories 210 may reside in various locations including servers 235. For example, a data repository used by server 235 may be local to server 235 or may be remote from server 235 and in communication with server 235 via a network-based or dedicated connection of network 220. Data repositories 210 (a) and 210 (b) may be of distinct types or of the same type. In certain examples, a data repository may be a database which is an organized collection of data stored and accessed electronically from one or more storage devices such as one or more servers 235. The one or more servers 235 may be configured to execute a database application that provides database services to other computer programs or to computing devices (e.g., client device 205 and analyzer 215) within the computing environment, as defined by a client-server model. One or more of these databases may be adapted to enable storage, update, and retrieval of data to and from the database in response to SQL-formatted commands or like programming language that is used to manage databases and perform various operations on the data within them.

The analyzer 215 comprises a set of tools 240 for analyzing and visualizing data (i.e., data stored in data repository 210). The analyzer 215 is used to execute a process for generating an accurate and high confident genotype from a patient at risk of, diagnosed with, or being treated for a genetic disease associated with or caused by changes to a tandem repeat region such as the polyTG/polyT region in the genome (e.g., cystic fibrosis). In the configuration depicted in FIG. 2, the set of tools 240 includes three subsystems: a variant simulator 245 (e.g., ART), a NGS analysis pipeline 250, and a clinical sample orchestrator 255. The variant simulator 245 is responsible for generating simulated truth variant samples (e.g., FASTQ read files, alignment files) from all combinations of polynucleotide repeats. For example, spinocerebellar ataxia type 2 is caused by an expansion of the CAG (cytosine-adenine-guanine) trinucleotide repeat in the ATXN1 gene, where affected individuals have a CAG expansion of greater than 34 repeats, with the most common expansion being between 37-39. Accordingly, variant simulator 245 can generate simulated truth variants of a CAG expansion comprising 34, 35, 36, 37, 38, 39, and 40 CAG repeats. As another nonlimiting example, variant simulator 245 may be used to generate simulated variants for the polyTG/polyT repeat tract in the CFTR gene. In this example, the parameters of variant simulator 245 can be set to generate simulated ground truth polyT and polyTG variants with predetermined, known, numbers of Ts and TGs ranging from any combination of TG:7-15 and T:1-12. To generate simulated variants, variant simulator 245 uses a set of simulation tools, or optimized algorithms, that consider technology-specific read error and base quality parameters. For example, Illumina sequencing uses a sequencing by synthesis (e.g., base-by-base) method, thus the main error type is base substitution. Variant simulator 245 can simulate base substitution errors for Illumina sequencing methods by considering the base quality score associated with the base call. Once generated, the simulated truth samples are used by the variant simulator 245 itself, the NGS analysis pipeline 250, and/or the clinical sample orchestrator 255.

The NGS analysis pipeline 250 takes as input simulated truth samples and/or clinical sequencing files to generate a genotype and corresponding fingerprint. To achieve this, the NGS analysis pipeline 250 executes a series of bioinformatic codes to process the simulated truth “sequencing files”. The bioinformatics codes may include scripts (i) to load the simulated truth sample (as either a FASTQ reads file or aligned file), (ii) perform quality filtering and/or adapter trimming, (iii) perform mapping and alignment, if a FASTQ reads file is loaded), (iv) perform sorting of alignment files, (v) base quality determination, (vi) variant calling, (vii) annotations, etc. Each script may be initiated by the output generated from the previous script so that all or the designated scripts run automatically. The NGS analysis pipeline outputs many variant tables (e.g., vcf, csv, and xml files), which include features (e.g., chromosome number and region, variant type, reference sequence, variant alteration, etc.) that describe each of the variants in the tables. To form a variant fingerprint for a simulated truth sample, the NGS analysis pipeline 250 can execute another bioinformatics code that extracts the desired features corresponding to the simulated truth sample from the variant table. The extracted features are concatenated into one string referred to as a mutation pattern or variant fingerprint. An exemplary mutation pattern could be 117188683_T_TGT_INS_HET, where 117188683 (on hg19 chromosome 7) represents the position of the base that is mutated, T indicates the reference sequence base at position 117188683, TGT indicates the mutation pattern, INS indicates the mutation type (e.g., insertion), and HET indicates the variant zygosity (e.g., heterozygous).

The clinical sample orchestrator 255 is responsible for accessing/loading clinical sample sequencing files from the data repositories 210 and initiating their processing by the NGS analysis pipeline 250 to generate a mutation finger print for the clinical sample and report the corresponding genotype variant (e.g., TG_11_with_T_05 and TG_11_with_T_07) and corresponding fingerprint for the clinical sample. In some instances, once a clinical sample is received, the NGS analysis pipeline 250 may either be manually initiated or automatically initiated by a bioinformatic script.

The analyzer 215 may reside in one or more locations including servers 235. For example, analyzer 215 used by server 235 may be local to server 235 or may be remote from server 235 (e.g., offered as an application or service as part of a cloud-based infrastructure) and in communication with server 235 via a network-based or dedicated connection of network 220. The analyzer 215 may be of different configurations or of the same configuration. The one or more servers 235 may be configured to execute a discovery application that provides discovery services to other computer programs or to computing devices (e.g., client device 205) within the computing environment, as defined by a client-server model.

In various instances, server 235 may be adapted to run one or more services or software applications that enable one or more embodiments described in this disclosure. In certain instances, server 235 may also provide other services or software applications that may include non-virtual and virtual environments. In some examples, these services may be offered as web-based or cloud services, such as under a Software as a Service (SaaS) model to the users of client device 205. Users operating client device 205 may in turn utilize one or more client applications to interact with server 235 to utilize the services provided by these components (e.g., database and rescue applications). In the configuration depicted in FIG. 2, server 235 may include one or more components 260, 265, and 270 that implement the functions performed by server 235. These components may include software components that may be executed by one or more processors, hardware components such as a motherboard, one or more processors, and memory, or combinations thereof. It should be appreciated that multiple different device configurations are possible, which may be different from computing environment 200. The example shown in FIG. 2 is thus one example of a computing environment (e.g., a distributed system for implementing an example computing system) and is not intended to be limiting.

Server 235 may be composed of one or more general purpose computers, specialized server computers (including, by way of example, PC (personal computer) servers, UNIX® servers, mid-range servers, mainframe computers, rack-mounted servers, etc.), server farms, server clusters, or any other appropriate arrangement and/or combination. Server 235 may include one or more virtual machines running virtual operating systems, or other computing architectures involving virtualization such as one or more flexible pools of logical storage devices that may be virtualized to maintain virtual storage devices for the server. In various instances, server 235 may be adapted to run one or more services or software applications that provide the functionality described in the foregoing disclosure.

The computing systems in server 235 may run one or more operating systems including any of those discussed above, as well as any commercially available server operating system. Server 235 may also run any of a variety of additional server applications and/or mid-tier applications, including HTTP (hypertext transport protocol) servers, FTP (file transfer protocol) servers, CGI (common gateway interface) servers, JAVA® servers, database servers, and the like. Exemplary database servers include without limitation those commercially available from Oracle®, Microsoft®, Sybase®, IBM® (International Business Machines), and the like.

In some implementations, server 235 may include one or more applications to analyze and consolidate data feeds and/or data updates received from users of client computing devices 205. As an example, data feeds and/or data updates may include, but are not limited to, in vivo feeds, in silico feeds, or real-time updates received from public studies, user studies, one or more third party information sources, and data streams (continuous, batch, or periodic), which may include real-time events related to sensor data applications, biological system monitoring, and the like. Server 235 may also include one or more applications to display the data feeds, data updates, and/or real-time events via one or more display devices of client computing devices 205.

Sequencer 275 is a sequencing device which is any machine capable of sequencing one or more nucleic acid molecules to generate raw sequencing data (e.g., reads). Library prepared nucleic acid samples may be pooled and loaded into lanes of a sequencing flow cell. The flow cell may be loaded into sequencer 275 and imaged to generate sequence data. For example, reagents that interact with the nucleic acid samples fluoresce at particular wavelengths in response to an excitation beam and thereby return a signal for imaging. For instance, the fluorescent components may be generated by fluorescently tagged nucleic acids that hybridize to complementary molecules of the components or to fluorescently tagged nucleotides that are incorporated into an oligonucleotide using a polymerase. As will be appreciated by those skilled in the art, the wavelength at which the dyes of the sample are excited and the wavelength at which they fluoresce will depend upon the absorption and emission spectra of the specific dyes. Sequencer 275 may optionally include or be operably coupled to its own dedicated sequencer computer with its own input/output mechanisms, one or more processors, and memory. Additionally or alternatively, sequencer 275 may be operably coupled to a server 235 or client device 205 via network 220. Client device 205 may access the raw sequencing data files from data repositories 210 and execute instructions for analyzing or communicating the sequence data to network 220.

### III. Sample Acquisition and Preparation

As described herein, “patient,” and “subject” are used interchangeably and refer to a mammal. In certain embodiments, subjects are “patients,” i.e., living humans that are receiving medical care for a disease or condition. The disease or condition may be a genetic disease associated with alterations in tandem repeat genomic regions, for example and without limitation, cystic fibrosis, Huntington's disease, Kennedy's disease, myotonic dystrophy, Fragile X syndrome, and spinocerebellar ataxia, CAG/polyglutamine diseases, polyalanine-encoding expansions, spinal and/or bulbar muscular atrophy, Friedreich ataxia, C9orf72 frontotemporal dementia, Unverricht-Lundborg myoclonic epilepsy, and the like. This includes persons with no defined illness who are being investigated for signs of disease. In some embodiments, the patient or subject may be at risk of/diagnosed with/being treated for cystic fibrosis.

In many instances, a clinical diagnosis for a particular disease (e.g., cystic fibrosis) or a treatment plan is made by genetically testing a sample collected from a patient at risk of/diagnosed with/being treated for a genetic disease. A sample can be a cell-containing liquid or a tissue comprising nucleic acid molecules. The sample can comprise, but is not limited to, amniotic fluid, tissue biopsies, blood, prenatal blood, blood cells, bone marrow, fine needle biopsy samples, peritoneal fluid, amniotic fluid, plasma, pleural fluid, saliva, semen, serum, tissue or tissue homogenates, frozen or paraffin sections of tissue. Methods of obtaining a sample include but are not limited to biofilms, aspirations, tissue sections, swabs, drawing blood or other fluids, surgical or needle biopsies, and the like. In various embodiments, the sample is a whole blood sample and/or a prenatal blood sample that is collected by blood draw from the patient for genetic testing of cystic fibrosis.

To prepare the clinical sample(s) for sequencing, nucleic acids (e.g., RNA and DNA) are isolated from the sample (e.g., blood). In several embodiments, DNA is isolated from blood samples. Various methods are known in the art for isolating DNA from a sample, such as using a reagent kit or by experimental means. A reagent kit (e.g., tubes and DNA extraction reagents, etc.) for library preparation may include materials such as probes for hybrid capture as well as any useful reagents & protocols for fragmentation, adapter ligation, purification/isolation, etc. Experimental methods for isolating/extracting DNA from a sample involve disruption and lysis of the starting material followed by the removal of proteins and other contaminants and finally recovery (e.g., alcohol precipitation) of the DNA.

In some instances, when it is determined that there is an insufficient amount of nucleic acid for analysis, amplification may be used to increase the amount of nucleic acid. Amplification refers to production of additional copies of a nucleic acid sequence and is generally carried out using polymerase chain reaction (PCR) or other technologies known in the art (e.g., Dieffenbach and Dveksler, PCR Primer, a Laboratory Manual, 1995, Cold Spring Harbor Press, Plainview, NY). PCR refers to methods by K. B. Mullis (U.S. Pat. Nos. 4,683,195 and 4,683,202, hereby incorporated by reference) for increasing concentration of a segment of a nucleic acid sequence in a mixture of genomic DNA without cloning or purification.

Following DNA isolation, the isolated DNA undergoes library preparation, which can include steps like fragmentation, end repair, adaptor ligation, enrichment, or any combination thereof. The isolated DNA are fragmented into a plurality of shorter double stranded DNA target fragments either physically (e.g., acoustic shearing, sonication) or enzymatically (e.g., with DNase I). Fragmentation, depending on the method, can generate DNA fragments ranging in size from 150 bp to 3 kb in length. Fragmentation can also cause short overhangs at the 3′ and 5′ ends of the fragments. Any end repair methods known in the art may be used, such as an exonuclease reaction that adds an adenosine to overhangs. Ligation-based library preparations often make use of an adapter design which can incorporate an index sequence (e.g., a sample index sequence to identify sample origin for a nucleic acid sequence) via a ligase or polymerase to the end repaired DNA fragments. Adapter oligonucleotides are often complementary to flow-cell anchors, and sometimes are utilized to immobilize a nucleic acid library to a solid support, such as the inside surface of a flow cell, for example. An adapter oligonucleotide may comprise an identifier, one or more sequencing primer hybridization sites (e.g., sequences complementary to universal sequencing primers, single end sequencing primers, paired end sequencing primers, multiplexed sequencing primers, and the like), or combinations thereof (e.g., adapter/sequencing, adapter/identifier, adapter/identifier/sequencing). Optionally, the DNA library, or parts thereof, are amplified (e.g., amplified by a PCR-based method). For example, a sequencing method may comprise amplification of a DNA library prior to or after immobilization on a bead or solid support (e.g., a solid support in a flow cell) using any suitable method. Library amplification is typically used as an enrichment step to increase the concentration of the library for sequencing.

### IV. Sequencing Methods

The library prepped nucleic acids are sequenced using a machine capable of sequencing nucleic acids (e.g., sequencer 275 described with respect to FIG. 2). Examples of sequencing may include, without limitation, NovaSeq, HiSeq, Genome Analyzer IIx, MiSeq, NextSeq, HiScanSQ, 454 DNA sequencer, GS FLX+, GS Junior System, OLID next-generation sequencing platform, Ion PGM System, Ion Proton System, Ion S5, Ion S5xl, CEQ 8000, RS system, Sequel system, nanopore sequencers, DNBSEQ-G50, DNBSEQ-G400, DNBSEQ-T7, Ultima Genomics UG100, etc. In certain instances, a full or substantially full sequence is obtained and sometimes a partial sequence is obtained.

Any suitable method of sequencing nucleic acids can be used, non-limiting examples of which include Maxim & Gilbert, chain-termination methods, sequencing by synthesis, sequencing by ligation, sequencing by mass spectrometry, microscopy-based techniques, the like or combinations thereof. In some embodiments, a first-generation technology, such as, for example, Sanger sequencing methods including automated Sanger sequencing methods, including microfluidic Sanger sequencing, can be used in a method provided herein. In some embodiments, sequencing technologies that include the use of nucleic acid imaging technologies (e.g., transmission electron microscopy (TEM) and atomic force microscopy (AFM)), can be used. In some embodiments, a high-throughput sequencing method is used. High-throughput sequencing methods generally involve clonally amplified DNA templates or single DNA molecules that are sequenced in a massively parallel fashion, sometimes within a flow cell. Next generation (e.g., 2^{nd} and 3^{rd} generation) sequencing techniques capable of sequencing DNA in a massively parallel fashion can be used for methods described herein and are collectively referred to herein as “massively parallel sequencing” (MPS). In certain embodiments, a non-targeted approach is used where most or all nucleic acids in a sample are sequenced, amplified and/or captured randomly.

Other suitable sequencing technologies may include single molecule, real-time (SMRT) technology of Pacific Biosciences (in SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW) where the fluorescent label is excited and produces a fluorescent signal, and the fluorescent tag is cleaved off. Detection of the corresponding fluorescence of the dye indicates which base was incorporated); nanopore sequencing (DNA is passed through a nanopore and each base is determined by changes in current across the pore, as described in Soni & Meller, 2007, Progress toward ultrafast DNA sequence using solid-state nanopores, ClinChem 53 (11): 1996-2001); chemical-sensitive field effect transistor (chemPET) array sequencing (e.g., as described in U.S. Pub. 2009/0026082); and electron microscope sequencing (as described, for example, by Moudrianakis, E. N. and Beer M., in Base sequence determination in nucleic acids with the electron microscope, III. Chemistry and microscopy of guanine-labeled DNA, PNAS 53:564-71 (1965).

### First Generation Sequencing: Sanger

Sanger sequencing is a first-generation DNA sequencing method that has long been considered the gold standard for the accurate detection of single nucleotide variants (SNVs). First generation sequencing techniques, like Sanger, utilize a chain-termination method wherein specialized DNA bases (dideoxynucleotides or ddNTPs) are randomly incorporated into a growing DNA chain of nucleotides (A, C, G, T) generating different length DNA fragments. Fragments are size separated by capillary electrophoresis and a laser is used to excite the unique fluorescence signal associated with each ddNTP. As the fluorescence signal is recorded, a chromatograph is generated, showing which base is present at a given location of the target region being sequenced. In the clinical setting, Sanger provides flexibility for testing single or small batch samples (no more than a 10 gene panel) for prenatal, carrier, and genetic testing and can provide results in a relatively short period of time. However, Sanger is limited to short DNA sequences, approximately 300-1,000 bases, and can be more expensive compared to newer generation sequencing methods.

### Second Generation Sequencing: Next Generation Sequencing

NGS, can be applied in various sequencing techniques that include whole-genome sequencing (WGS), whole-exome sequencing (WES), targeted genome sequencing (TGS), and any other sequencing technique known to one skilled in the art may be performed on the prepared DNA library samples. NGS is based on the amplification of DNA on a solid surface using fold-back PCR and anchored primers. DNA is fragmented, and adapters are added to the 5′ and 3′ ends of the fragments. DNA fragments that are attached to the surface of flow cell channels are extended, bridge amplified, and denatured. Primers, DNA polymerase and four fluorophore-labeled, reversibly terminating nucleotides are used to perform sequential sequencing. After nucleotide incorporation, a laser is used to excite the fluorophores, an image is captured, and the identity of the first base is recorded. The 3′ terminators and fluorophores from each incorporated base are removed and the incorporation, detection and identification steps are repeated. Multiple cycles of the solid-phase amplification followed by denaturation can create several million clusters of approximately 1,000 copies of single-stranded DNA molecules of the same template in each channel of the flow cell. Sequencing according to this technology is described in U.S. Pat. Nos. 7,960,120; 7,835,871; 7,232,656; 7,598,035; 6,911,345; 6,833,246; 6,828,100; 6,306,597; 6,210,891; U.S. Pub. 2011/0009278; U.S. Pub. 2007/0114362; U.S. Pub. 2006/0292611; and U.S. Pub. 2006/0024681, each of which are incorporated by reference in their entirety.

Sequencing methods (e.g., NGS and Sanger) generate a large number of reads. As used herein, “reads” (e.g., “a read,” “a sequence read”) are short nucleotide sequences produced by any sequencing process described herein or known in the art. Reads can be generated from one end of nucleic acid fragments (“single-end reads”), and sometimes are generated from both ends of nucleic acid fragments (e.g., paired-end reads, double-end reads). The length of a sequence read is often associated with the particular sequencing technology. High-throughput methods, for example, provide sequence reads that can vary in size from tens to hundreds of base pairs (bp). Sequencing reads may have a mean, median, average, or absolute length of about 15 bp to about 1000 bp. For example, sequencing reads may be about 15 bp, 16 bp, 17 bp, 18 bp, 19 bp, 20 bp, 25 bp, 50 bp, 100 bp, 150 bp, 200 bp, 300 bp, 400 bp, 500 bp, 600 bp, 700 bp, 800 bp, 900 bp, or about 1000 bp or about any integer value between 15 bp and 1000 bp. Sequencing reads, and their associated quality scores, are stored in files known as FASTQ files or FASTA files. Typically, FASTQ files can comprise about 1 million to about 5 million reads per sample; however, more or less reads may be generated depending on the sample.

### Bioinformatics Workflow

In some embodiments, sequence reads are generated, obtained, gathered, assembled, manipulated, transformed, processed, and/or provided by a sequence subsystem comprising a sequencer (e.g., sequencer 275 described with respect to FIG. 2). A machine comprising a sequence subsystem can be a suitable machine and/or apparatus that determines the sequence of a nucleic acid utilizing a sequencing technology known in the art. In some embodiments a sequence subsystem can align, assemble, fragment, complement, reverse complement, and/or error check (e.g., error correct sequence reads). The sequence reads are processed using a sequence processing subsystem to obtain sequence read data. The processing of the sequence reads includes read alignment, mapping, and filtering. To perform all these processing steps, the bioinformatics workflow can comprise any combination of bioinformatic techniques including demultiplexing, reference genome alignment, database design, and variant calling as nonlimiting examples.

As described above, the outputs of sequencing are FASTQ files that comprise all the reads for a single sample. Part of the process of generating FASTQ files is demultiplexing (e.g., sorting) all the different library samples that were pooled together in a single flow cell lane into their own FASTQ file. In a typical sequencing run, multiple library samples (e.g., 4, 12, 16, etc.) are combined and loaded onto a single lane of a sequencing flow cell. Because during the library preparation, each DNA fragment in a sample had a corresponding unique barcode ligated onto the fragments. Accordingly, when multiple libraries are pooled for sequencing, the barcodes allow for the samples to be distinguished from one another. The barcodes are also what are used to sort each sample into its own sequencing FASTQ file (i.e., demultiplexing).

Alignment of reads to a reference genome (e.g., a human reference genome) involves mapping any number of reads to a specified nucleic acid region (e.g., a chromosome or portion thereof) and are referred to as counts. As used herein, the term “reference genome” can refer to any known, sequenced or characterized genome, whether partial or complete, of any organism or virus which may be used to reference identified sequences from a subject. Suitable reference human genomes may include a published human genome (e.g., hg19 or hg38) or some other reference material, such as “gold standard” sequences obtained by, e.g., Sanger sequencing of subject nucleic acid.

Any suitable mapping/alignment method (e.g., process, algorithm, program, software, subsystem, the like or combination thereof) can be used. Non-limiting examples of computer algorithms that can be used to align sequences include, without limitation, BLAST, BLITZ, FASTA, BOWTIE 1, BOWTIE 2, ELAND, MAQ, PROBEMATCH, SOAP, BWA or SEQMAP, or variations thereof or combinations thereof. The terms “aligned,” “alignment,” or “aligning” generally refer to two or more nucleic acid sequences that can be identified as a match (e.g., 100% identity) or partial match. Alignments can be done manually or by a computer (e.g., a software, program, subsystem, or algorithm), non-limiting examples of which include the Efficient Local Alignment of Nucleotide Data (ELAND) computer program distributed as part of the Illumina Genomics Analysis pipeline. Alignment of a sequence read can be a 100% sequence match. In some cases, an alignment is less than a 100% sequence match (i.e., non-perfect match, partial match, partial alignment). In some embodiments an alignment is about a 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76% or 75% match. In some embodiments, an alignment comprises a mismatch. In some embodiments, an alignment comprises 1, 2, 3, 4 or 5 mismatches. Two or more sequences can be aligned using either strand (e.g., sense or antisense strand). In certain embodiments a nucleic acid sequence is aligned with the reverse complement of another nucleic acid sequence. The results from alignment are deposited in an alignment file (e.g., BAM).

As a quality control step, all alignment files may be filtered to remove non-primary alignment records, reads mapped to improper pairs, and reads with more than six edits. Individual bases are excluded if their Phred base quality is less than 30 in tumor samples and less than 20 in normal samples. As described herein, the term “less than” comprises all whole numbers and rational numbers. For example, less than 30 includes 29.9, 29.8, 29.7, 29.6, 29.5, 29.4, 29.3, 29.2, 29.1, 29.0, 25, 20, 15, 10, 5, and 0.

During reference genome alignment, variations between the sample and the reference genome may be identified. The process of comparing sequence data to a reference is called variant calling. As described herein, variants comprise naturally occurring alterations to a DNA sequence not found in the reference sequence, and the alterations can be classified as benign, likely benign, variant of unknown significance, likely pathogenic or pathogenic. Moreover, variants can comprise both germline variants (e.g., variants present in all the body's cells) and somatic variants (variants that arise during the lifetime of an individual, such as if an individual develops cancer). Examples of variants include small sequence variants (less than 50 base pairs) such as single nucleotide variants (SNVs), single nucleotide polymorphisms (SNPs) and small structural variants (SVs) (e.g., deletions, insertions, insertions and deletions, sometimes referred to as indels) and larger (greater than 50 base pairs) SVs such as chromosomal rearrangements (e.g., translocations and inversions) and copy number changes. SNVs/SNPs are the result of single point mutations that can cause synonymous changes (nucleotide change does not alter the encoded amino acid), missense changes (nucleotide change does alter the encoded amino acid), or nonsense changes (resulting amino acid change converts the encoded codon to a stop codon). Further, variants can occur in both coding and non-coding regions of the genome and can be detected by WGS, as opposed to targeted gene panels, and target specific probes.

Variant calling can use one or more variant calling tools to examine the aligned/mapped sequencing data and reference genome side-by-side to determine the existence of sequence mutations (single base changes and small indels). The variant calling tool may extract candidate variants from alignment data, score a number of individual metrics for each variant, and apply these scores both individually and in combination to identify bona fide sequence mutations and to exclude sequence artifacts. Any suitable technique/variant calling tool may be used to detect structural alterations such as, for example, MuTect, Strelka, dysgu, and/or JointSNVMix2. The list of detected variants and their properties (e.g., type of variant) are annotated and deposited in a variant file (e.g., variant call format (VCF)). The output VCF files can include about 1,500 to a few million variants; however, more or less variants may be found depending on the sample.

### V. Method for Generating Sequencing Fingerprint Profile

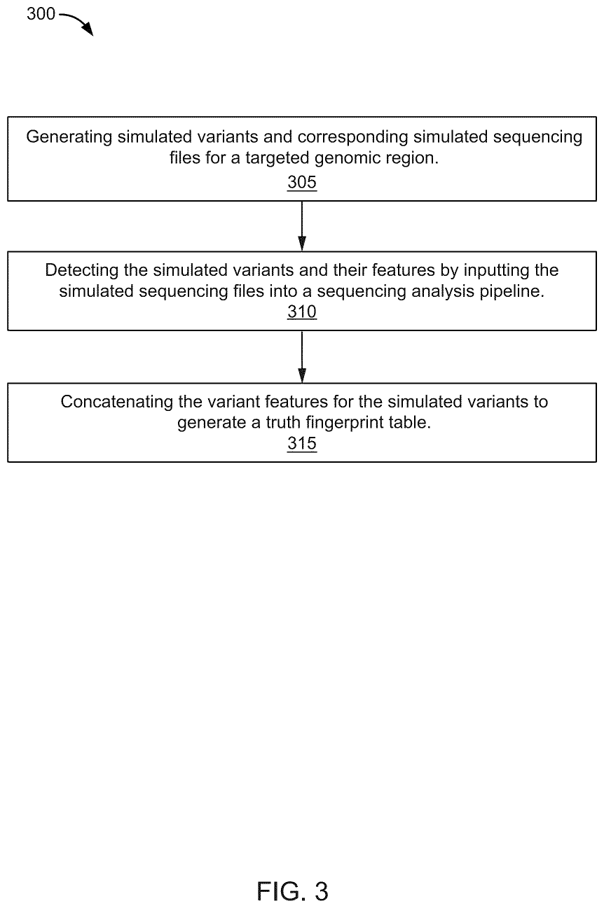

FIG. 3 shows an exemplary workflow for generating ground truth NGS-based fingerprint profiles for a set of simulated variants. The processing depicted in FIG. 3 may be implemented in software (e.g., code, instructions, program) executed by one or more processing units (e.g., processors, cores) of the respective systems, hardware, or combinations thereof (e.g., computing environment 200 described with respect to FIG. 2). The software may be stored on a non-transitory storage medium (e.g., on a memory device). The method presented in FIG. 3 and described below is intended to be illustrative and non-limiting. Although FIG. 3 depicts the various processing steps occurring in a particular sequence or order, this is not intended to be limiting.

At box 305, a set of simulated truth variants and corresponding simulated sequencing read files for a targeted genomic region (also referred to herein as target repeat genomic location, or a repeat tract) are generated in silico using a NGS simulation tool (e.g., variant simulator 245 described with respect to FIG. 2). The targeted genomic region may be a tandem repeat genomic region, and more specifically, ±500 base pairs of the CFTR polyTG/polyT region. Although the systems and techniques described herein are explained specifically with respect to the polyTG/polyT track genomic region, the systems and techniques would also be applicable to other tandem repeat genomic regions of a given genome. The NGS simulation tool is a synthetic next-generation sequencing reads generation tool that is used to create simulated truth variant samples computationally from all combinations of base pairs. As described herein, “all combinations” includes (i) the homozygous wild-type combination where neither allele comprises at least one variants, (ii) heterozygous combinations where one allele is wild-type and the other allele comprises at least one variant, and (iii) homozygous mutant combinations where both alleles comprise at least one variant that may be identical, different, or any combination thereof. The number of variants and read files that the simulation tool can generate for a genomic region depends on several factors, including the specific parameters set by the user, the length of the genomic region, and the complexity of the simulation. For example, the size of the genomic region being simulated (e.g., a portion of a gene, a single gene, a chromosome, or the entire genome) will significantly impact the number of variants. The mutation rate set in the simulation determines how frequently variants occur. Higher mutation rates will produce more variants. Additionally, the simulation tool might be able to generate various types of variants, including single nucleotide polymorphisms (SNPs), insertions, deletions (indels), and structural variants. Each type can occur at different frequencies. Parameters such as the number of haplotypes, the recombination rate, and the demographic model (e.g., population size changes) can also influence the number and distribution of variants. Lastly, the number of read files will depend on the sequencing coverage, read length, and the sequencing technology being simulated (e.g., Illumina, PacBio, Oxford Nanopore). Nonetheless, in certain embodiments-particularly those associated with the polyTG/polyT track genomic region—it is expected that a few hundred variants to a few thousand variants are generated (e.g., greater than 100 variants, greater than 1,000 variants, or greater than 5,000 variants, (e.g., at least 5,000 sets of simulated variants and corresponding simulated sequencing read files)).

In certain embodiments, the NGS simulation tool is a set of computational tools, for example, a variant simulation tool such as Bamsurgeon, which is configured to introduce variants into a read alignment file (such as BAM, SAM, CRAM) anchored on a reference genome (e.g., hg19, hg38), and a sequence read simulation tool such as wigsim, ART, VarSim, DWGSIM, InSilicoSeq, MASON, NEAT, Sim-it, which are configured to generate synthetic next-generation sequencing reads from a user-provided reference genome. Similar tools for PacBio, Oxford Nanopore and other long reads technologies are also available, and examples include PBSIM, SimLord and NPBSS. The NGS simulation tool generates synthetic sequencing reads by mimicking real sequencing process with empirical mutation and/or error models or quality profiles summarized from large, recalibrated sequencing data. The NGS simulation tool can also create simulated reads using a user's own read mutation and/or error model or quality profiles. The NGS simulation tool supports simulation of single-end, paired-end/mate-pair reads of one or more commercial next-generation sequencing platforms, for example, Illumina's Solexa, Roche's 454 and Applied Biosystems' SOLID.

The NGS simulation tool (e.g., set of computation tools) operate by initially taking as input a reference genome sequence (e.g., a reference genome sequence comprising the polyTG/polyT track genomic region) in a given data format, such as a FASTA format. This reference genome serves as the template from which variants are introduced and thereafter simulated reads are generated. Parameters are set for the tool(s) such as specifying the desired sequencing coverage (e.g., 100× coverage), defining the length of the reads and fragment size, choosing the sequencing platform to simulate, and specifying the mutation rates and/or error profiles that mimic real world mutations and/or error characteristics of the chosen sequencing technology. A variant simulation tool (e.g., Bamsurgeon, ART, wgsim, or VarSim) and/or a sequence read simulation tool (e.g., wigsim, ART, VarSim, DWGSIM, InSilicoSeq, MASON, NEAT, Sim-it) are then used to generate new BAM files or new fastq files with the desired mutation(s), specified coverage and read length. These tools can create various mutations including single nucleotide polymorphisms (SNPs), insertions, deletions, and structural variants. The tool(s) introduce mutations and/or errors based on the chosen profile to create a realistic simulation of sequencing reads. For paired-end reads, the tool(s) generate two reads per fragment, with a specified insert size distribution. This approach allows for the computing environment to create read files that reflect the presence of specific variants (i.e., a set of simulated truth variants and corresponding simulated sequencing read files).

As can be appreciated by a person skilled in the art, the sets of simulated truth variants may be generated for any genomic region of interest for any genome. As used herein, a tandem repeat tract is a genomic region of sequentially repeated DNA motifs that range in length from 1 to 10 nucleotides and repeat at least 5 times. For example, repeat lengths can include mono-nucleotide, di-nucleotides, tri-nucleotides, tetra-nucleotides, penta-nucleotides, hexa-nucleotides, hepta-nucleotides, octa-nucleotides, nona-nucleotides, and deca-nucleotides. Examples of tandem repeat genomic regions can include homopolymer sequences, microsatellite regions, macrosatellite regions, and the like.

Each set of simulated variants in the sets of simulated variants comprise a first allele with zero or more mutations, and a second allele with zero or more mutations. More specifically, each simulated haplotype may have a label of 0, 1, and/or 2. “0” indicates a wild-type allele (i.e., no variants are detected/present). “1” indicates one or more mutations are detected/present in the polyT OR polyTG region(s) and “2” indicates one or more mutations are detected/present in the polyT AND polyTG regions. The at least one simulated mutation in the first allele may be the same mutation(s), different mutation(s), or any combination thereof compared to the at least one simulated mutation in the second allele. By way of example, without limitation, the first allele and second allele may comprise the same first mutation in the same location and would be considered homozygous for the first mutation. Additionally or alternatively, the first allele may comprise a second mutation in a location and the second allele comprises the wild type sequence (e.g., no mutation) in the same location. This location is considered heterozygous with respect to the first allele.

The simulated sequencing read files comprise (i) a forward sequencing read file comprising at least 10,000 simulated reads, (ii) a reverse sequencing read file comprising at least 10,000 simulated reads, or both (i) and (ii). In other words, the simulated sequencing read files may comprise single-end sequencing reads (e.g., either a forward sequencing read file or a reverse sequencing read file), where the single sequencing read file comprises at least 10,000 simulated reads. As another example, the simulated sequencing read files may comprise pair-end sequencing reads (e.g., a forward sequencing read file and a reverse sequencing read file), where the forward and reverse read files, each comprise at least 10,000 reads for a total of at least 20,000 reads.

In some embodiments, the targeted genomic region has flanking regions with a predetermined number of base pairs (e.g., ±500 base pairs) of the CFTR polyTG/polyT region (e.g., chr7:117188660-117188689 in hg19/GRCh37). The NGS simulation tool is used to generate a set of simulated variant samples from all combinations of TG:7-15 and T:1-12, as well as their corresponding simulated sequencing read files for each simulated variant. Parameters for the simulation tool can be set by a user based on the needs of the experiment. Parameters may include fragment length, read length, coverage, and other parameters specific to the sequencing platform. By way of example, and not limitation, the parameters may include a fragment length between 150-500 bp, a read length between 100-300 bp, coverage of about 50× to about 150×, and additional parameters specific for the HiSeq 2500 platform. Each set of simulated variants comprises a first allele with zero or more mutations and a second allele with zero or more mutations. Further, the simulated sequencing read files comprise (i) a forward sequencing read file comprising at least 10,000 simulated reads, (ii) a reverse sequencing read file comprising at least 10,000 simulated reads, or both (i) and (ii). In total, multiple (e.g., 13,689) paired simulated read samples with two phased haplotypes, except cases where the two haplotypes are identical, are generated.

Implementation of the simulation tool to generate simulated variants and synthetic sequencing read files overcomes several challenges faced using standard experimentation methods. First, for every simulated variant, the number of Ts and TGs are known, thus the variant truth is known. Second, high quality sequencing reads are generated ensuring sufficient read coverage, reduced base call ambiguity, and high read alignment for the targeted genomic region (e.g., a targeted tandem repeat genomic region), as opposed to standard NGS methods. Third, high confident variant calls are made ensuring that the sequencing fingerprint profile is a robust, accurate, and reliable representation of its corresponding simulated variant, as opposed to NGS methods. Fourth, all variants and sequencing files are generated in silico, eliminating the use and cost associated with reagents and sequencing methods.

At box 310, the simulated sequencing read files are input into a sequencing analysis pipeline for variant detection. The sequencing analysis pipeline is a NGS analysis pipeline (e.g., NGS pipeline 250 described with respect to FIG. 2) designed to align sequencing read data (e.g., simulated read files or non-simulated read files) to a reference genome and to identify zero or more variations between the sequencing read data (e.g., simulated read files or non-simulated read files) and the reference genome. As described above, alignment of reads to a reference genome (e.g., a human reference genome) involves mapping any number of reads (e.g., simulated and/or non-simulated) to their originating nucleic acid region (e.g., a chromosome or portion thereof) and are referred to as counts. The alignment files are used by a variant calling tool to identify variations between the sequencing file (e.g., an alignment file) and the reference genome. There are different quality metrics associated with each variant that are used to assign a call status (e.g., pass or fail). Examples of quality metrics that are considered for variant calling include: coverage (i.e., the number of reads aligned against the variant position), quality score (i.e., estimated calculation by the variant calling algorithm indicating how confident the caller is in the variant call), phred-score, clustered events, duplicate, fragment, multiallelic, n ratio, orientation, position, slippage, haplotype, map quality, base quality, contamination, allele frequency, strand bias, etc. After variant calling, the detected variants are output into a variant file/table (vcf, xml, and the like) along with variant features that describe the variant. Variant features associated with the variant can include, without limitation, chromosome number, position (i.e., the co-ordinates of the variant), an identifier, reference allele (e.g., nucleic acid base from the reference genome), alternative allele (e.g., nucleic acid base from the sample), quality score, filter (e.g., pass/fail), variant type (insertion, deletion, SNV, etc.), zygosity, and the like.

At box 315, the variant features for the zero or more detected variants in the first allele and the variant features for the zero or more detected variants in the second allele are concatenated into a single string to generate a sequencing fingerprint profile. The concatenation process may be facilitated by a bioinformatics code that extracts the desired features corresponding to the simulated sample from the variant table and formats the sequencing fingerprint profile into the single string. The sequencing fingerprint profile comprises the starting coordinate for the variant, the reference genome nucleic acid base, the alternative nucleic acid base, the variant type, the zygosity of the variant, or any combination thereof. In various instances, a set of simulated variants may include more than one variant, indicating the sequencing fingerprint profile associated with such sets of simulated variants can include more than two fingerprint components. For example, a whole fingerprint can include 2, 3, 4, 5, 6 or more individual fingerprint components based on the number of variants introduced into the set of simulated variants. By way of example, the first simulated variant is TG(10)T(9) and the second simulated variant is TG(13)T(5). The concatenated feature string for this set of simulated variants has the following sequencing fingerprint profile (also referred to herein as a mutation pattern): 117188682_G_T_SNV_HET; 117188683_T_TGT_INS_HET, 117188684 T_G_SNV_HET; based on the alignment and variant calls generated by the sequencing analysis pipeline. Accordingly, the sequencing fingerprint profile is an NGS-based variant fingerprint profile. The exemplary variant fingerprint profile is interpreted as follows: For the set of simulated variants TG(10)T(9)_TG(13)T(7) the following features are known: the first allele (TG(10)T(9)) comprises (i) the 117188682_G_T_SNV_HET mutation pattern indicating that at base pair 117188682 the reference genome has a G, but the alternative allele is a T(i.e., G→T), indicating a SNV variant type. The zygosity is indicated as heterozygous because the G to T SNV was not identified on the second allele. As shown in Table 1, and indicated by the bold/italic T at position 82 for A1 (allele 1), the SNV caused the polyTG tract to be reduced by one unit (to TG(10)) and the polyT tract to increase by two (to T(9)). The second allele (A2 in Table 1) (TG(13)T(5)) comprises (i) the 117188683_T_TGT_INS_HET mutation pattern indicating that at base pair 117188683, the reference genome is a T, but the alternative allele includes an additional TG, indicating the variant type is an insertion, causing the polyTG track length to increase by one; and (ii) the 117188684_T_G_SNV_HET mutation, further increasing the poly TG track by one, bring the final polyTG length to 13, while reducing the polyT track length by two (TT→TG) from seven to five.

TABLE 1

| | Chr. | | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|---|
| | 61 | 62 | 63 | 64 | 65 | 66 | 67 | 68 | 69 | 70 | 71 | 72 | 73 | 74 | 75 | 76 | 77 | 78 | 79 | 80 | 81 | 82 | 83 | | 84 | 85 | 86 | 87 | 88 | 89 |
| | 1 | | 2 | | 3 | | 4 | | 5 | | 6 | | 7 | | 8 | | 9 | | 10 | | 11 | | 1 | | 2 | 3 | 4 | 5 | 6 | 7 |
| WT | T | G | T | G | T | G | T | G | T | G | T | G | T | G | T | G | T | G | T | | GT | | GT | | | T | T | T | T | T |
| A1 | T | G | GT | | GT | G | T | | GT | | GT | | GT | | GT | G | T | G | T | G | T | T | T | | T | T | T | T | T | T |
| A2 | T | G | GT | | GT | | GT | | GT | | GT | G | T | | GT | | GT | | GT | | GT | | GT | GT | G | T | T | T | T | T |

The simulated variant sets and their corresponding sequencing fingerprint profiles are then used as pairs to generate a truth fingerprint table. Part of the table generation process may further include performing deduplication on the sequencing fingerprint profiles to remove simulated variants from the truth fingerprint table that generate the same sequencing fingerprint profile. In other words, multiple simulated variants can have the same sequencing fingerprint profile. Simulated variant samples that generate the same mutation pattern are concatenated together so that a single simulated variant entry (comprising two or more simulated variant names) outputs the sequencing fingerprint profile. Overall, the truth fingerprint table comprises nearly every possible variant, within the scope of the parameters used for the simulation tool, that may be detected in the targeted genomic region (e.g., the poly TG/polyT tract of the CFTR gene) along with their corresponding sequencing fingerprint profile. The truth fingerprint table is output and may be deployed into an environment (e.g., a computational, production, and the like such as computing environment 200 described with respect to FIG. 2) where it is used to confirm the variant pattern and associated fingerprint profile of a clinical sample. The environment may be a clinical or research lab that is part of a medical institute that performs genetic testing. Construction and implementation of the truth fingerprint table eliminates having to perform Sanger sequencing to confirm the detected variant(s) in hard to sequence regions. Elimination of Sanger from the variant detection/confirmation workflow, results in a reduction of computer memory required to store the sequencing data, a reduction in use and cost of reagents, and an increase in turnaround time for getting results back to the clinician and patient.

### VI. Deployment of Truth-Fingerprint Table in a Clinical Setting

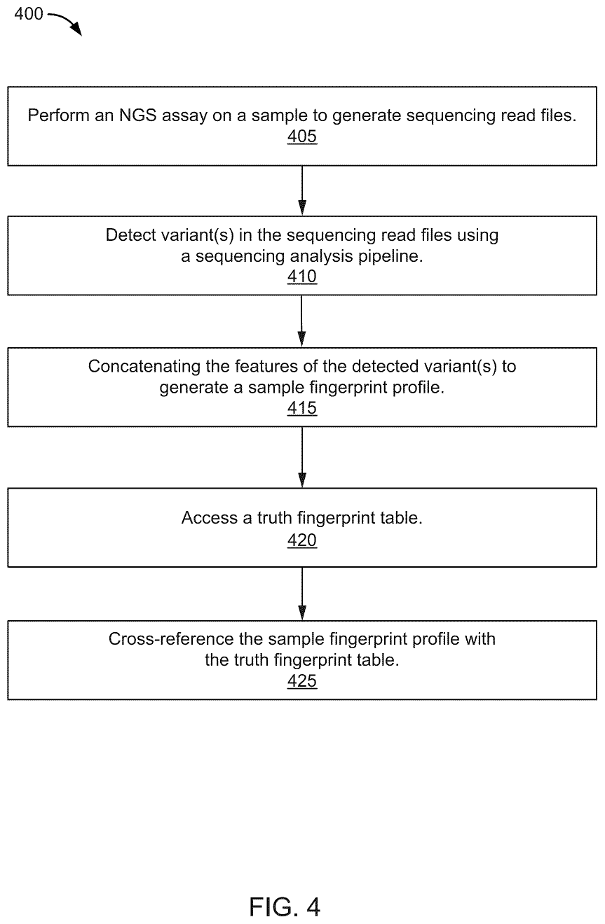

FIG. 4 shows an exemplary workflow for deploying the truth fingerprint table in a clinical setting. The processing depicted in FIG. 4 may be implemented in software (e.g., code, instructions, program) executed by one or more processing units (e.g., processors, cores) of the respective systems, hardware, or combinations thereof (e.g., computing environment 200 described with respect to FIG. 2). The software may be stored on a non-transitory storage medium (e.g., on a memory device). The method presented in FIG. 4 and described below is intended to be illustrative and non-limiting. Although FIG. 4 depicts the various processing steps occurring in a particular sequence or order, this is not intended to be limiting.

At box 405, a NGS assay (e.g., WGS, WES, TGS) is performed on a sample to generate sequencing read files. The sample is collected from a subject or patient who may be at risk of/diagnosed with/being treated for a genetic disease associated with a tandem repeat genomic region. In some embodiments, the sample is collected from a subject or patient at risk of/diagnosed with/being treated for cystic fibrosis. The sample is a cell-containing liquid or a tissue comprising nucleic acid molecules (e.g., DNA or RNA). In various embodiments, the sample is a whole blood sample and/or a prenatal blood sample that is collected by blood draw from the patient for genetic testing of cystic fibrosis.

Once a sample is collected, the sample is prepared for sequencing using either NGS or Sanger sequencing methods. The sample preparations, library preparations, and sequencing methods described above are all considered for sample and library preparations and sequencing. Briefly, sample and library preparation can include nucleic acid isolation, fragmentation, end repair, adaptor ligation, enrichment, or any combination thereof. The library prepared sample undergoes sequencing using an appropriate sequencing device (e.g., sequencer 275 described with respect to FIG. 2) to generate sequencing files (e.g., FASTQ, FASTA, etc.). The sequencing files comprise (i) a forward sequencing read file, (ii) a reverse sequencing read file, or both (i) and (ii). In other words, the sequencing read files may comprise single-end sequencing reads (e.g., either a forward sequencing read file, or a reverse sequencing read file), where the single sequencing read file comprises at least 0.5 million reads. As another example, the sequencing read files may comprise pair-end sequencing reads (e.g., a forward sequencing read file and a reverse sequencing read file), where the forward and reverse read files each comprise at least 0.5 million reads for a total of at least 1 million reads. Following sequencing, the sequencing read files may be stored in a data storage device (e.g., data repository 210 described with respect to FIG. 2) for long term storage and/or to be accessed for downstream processing.

The NGS assay can include WGS, WES, and/or TGS, depending on the nature of the genetic test. In some embodiments, the NGS assay is a targeted NGS that generates sequencing reads covering a genomic region±a predetermined number of base pairs (e.g., ±500 base pairs) of a tandem repeat genomic region. The tandem repeat genomic region may be regions associated with a genetic disease or condition. By way of example, and without limitation, these genomic regions include cystic fibrosis, Huntington's disease, Kennedy's disease, myotonic dystrophy, Fragile X syndrome, and spinocerebellar ataxia, CAG/polyglutamine diseases, polyalanine-encoding expansions, spinal and/or bulbar muscular atrophy, Friedreich ataxia, C9orf72 frontotemporal dementia, Unverricht-Lundborg myoclonic epilepsy, and the like. In certain embodiments, the targeted NGS assay generates sequencing reads covering a genomic region±a predetermined number of base pairs (e.g., ±500 base pairs) of polyTG/polyT region of the CFTR gene (e.g., chr7:117188660-117188689 in hg19/GRCh37).

At box 410, the sequencing read files are processed using a sequencing analysis pipeline (e.g., NGS analysis pipeline as described with respect to FIG. 2) for variant detection. Processing comprises (i) inputting the sequencing read files into the sequencing analysis pipeline for alignment to a reference genome, (ii) identifying zero or more variations between the sequencing read files and the reference genome (e.g., variant calling), (iii) and outputting a variant table comprising the zero or more detected variants and their corresponding variant features. Variant calling uses a variant caller algorithm to detect and assess variations observed between an experimental sample and a reference genome (e.g., a human reference genome). In some instances, one or more variants may not be detected, indicating the sample does not comprise a mutation (i.e., comprises two wild type alleles) in the targeted tandem repeat genomic region. Such a result indicates that the patient does not have the disease (e.g., cystic fibrosis) and other genetic tests may need to be performed. In other instances, one or more variants are detected and reported out in the variant table, indicating the patient is at risk of and/or is diagnosed with a disease (e.g., cystic fibrosis). The variant features that are reported out with the one or more detected variant(s) describe the quality, genomic location, and characteristics of the detected variant(s). Features can include chromosome number, position (i.e., the co-ordinates of the variant), an identifier, reference allele (e.g., nucleic acid base from the reference genome), alternative allele (e.g., nucleic acid base from the sample), quality score, filter (e.g., pass/fail), variant type (insertion, deletion, SNV, etc.), zygosity, and the like.

At box 415, the variant features for each of the one or more detected variants are concatenated into a single string to generate a sample fingerprint profile. The concatenation process may be facilitated by a bioinformatics code that extracts the desired features corresponding to the simulated sample from the variant table and formats the sequencing fingerprint profile into the single string. The sequencing fingerprint profile comprises the starting coordinate for the variant, the reference genome nucleic acid base, the alternative nucleic acid base, the variant type, the zygosity of the variant, or any combination thereof. Because the fingerprint profile is generated from the variant features of the targeted tandem repeat genomic region, it is specific to the variant that occurred in that particular region.

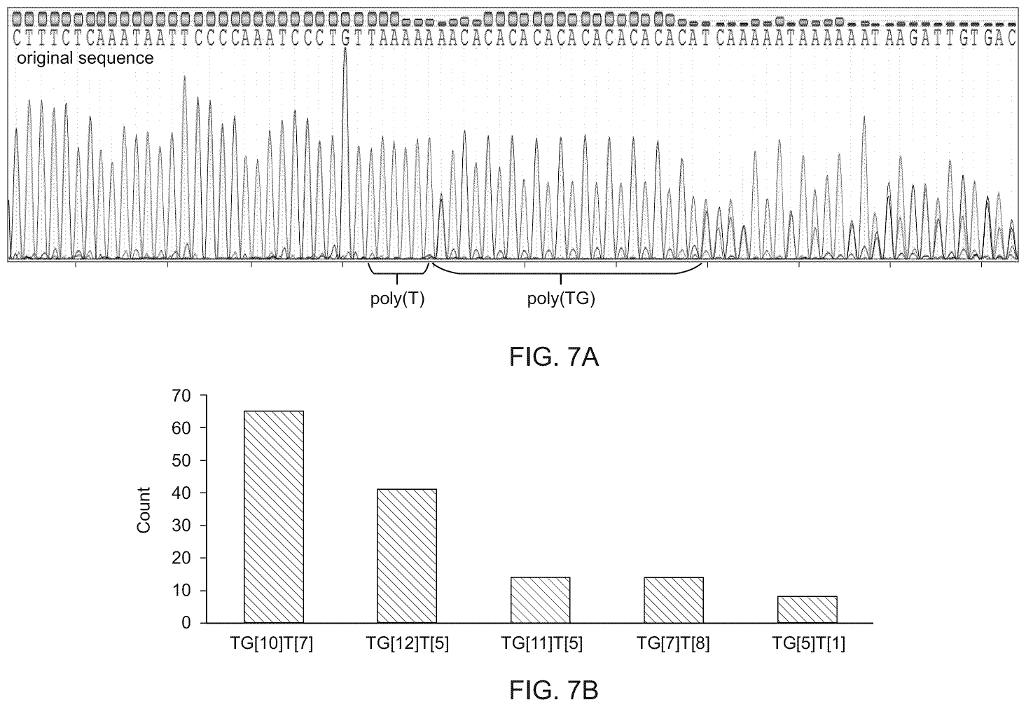

In some cases, the fingerprint profile table alone may be unable to resolve or make definitive polyTG/polyT variant calls due to a variety of factors, such as insufficient read coverage, poor mapping/alignment quality, poor base quality, and the like. When such a case occurs, the analysis tool (e.g., analysis tool 215 described with respect to FIG. 2) accesses the sequencing read files and counts the number of reads that align to the location of the variant in question to resolve ambiguous variant calls. The use of aligned read counts is also applied to situations where more than one variant set have the same sample fingerprint profile. The read count can distinguish between the variant sets, based on read distribution to determine which variant is the true variant of that location (refer to FIG. 7B for a graphical representation of this method).

At box 420, a truth fingerprint table is accessed that comprises pairs of simulated variants (e.g., at least 5,000 pairs of simulated variants) and their corresponding sequencing fingerprint profile. The simulated variant-sequencing fingerprint profile pairs are high confident, ground truth “labels” for nearly every possible variant combination that could occur in the targeted tandem repeat region (e.g., the same targeted tandem repeat region as the targeted NGS assay performed on the patient sample). Accordingly, the sequencing fingerprint profile also serves as a high confident, ground truth “label” for its corresponding simulated variant indicating the NGS-based variant calling profile that is expected to be generated for that specific set of variants. As described herein, “nearly every possible combination” refers to all possible combinations that can be generated based on the parameters set for the simulation tool. Further, combinations include (i) the homozygous wild-type combination where neither allele comprises a variant, (ii) heterozygous combinations where one allele is wild-type and the other allele comprises at least one variant, and (iii) homozygous mutant combinations where both alleles comprise at least one variant that may be identical, different, or any combination thereof.

At box 425, the sample fingerprint profile is cross-referenced with the sequencing fingerprint profiles in the truth fingerprint table to determine whether a match exists. When a match exists between the sample fingerprint profile and the sequencing fingerprint profiles in the truth fingerprint table, a report is generated comprising the sample fingerprint profile and the detected variant in the sample. If a sample fingerprint profile matches more than one sequencing fingerprint profiles, then the read profile (FIG. 7B) is checked to make the final polyTG/polyT calls. When a match does not exist between the sample fingerprint profile and the sequencing fingerprint profiles in the truth fingerprint table, (i) the truth fingerprint table is updated to include the sample fingerprint profile, (ii) cross-referencing is repeated for the sample fingerprint table, where a match now exists, and (iii) a report is generated comprising the sample fingerprint profile and the detected variant in the sample. Because the truth fingerprint table comprises high confident, ground truth “labels,” Sanger sequencing to confirm the detected variant(s) is not required when a match is or is not found. By eliminating the Sanger confirmation step, less overall computer memory is required to store the sequencing data, costs associated with running Sanger are eliminated, and the delay of getting results back to clinicians and/or patients is also eliminated. Moreover, further support that Sanger sequencing confirmation may be eliminated is provided in Example 4, specifically FIG. 7B, which demonstrates that the integration of in silico generated high confident, ground truth pairs of variant sets-sequencing fingerprint profiles to determine/confirm variant calls of clinical samples produces 100% concordance with Sanger confirmatory testing.

As described herein, a “match” between a sample fingerprint profile and the sequencing fingerprint profiles in the truth fingerprint table refers to an exact match. If an exact match is not found, the sample fingerprint profile and its corresponding variant set are added to the truth fingerprint profile and indicate a new variant detected in the targeted tandem repeat genomic region.

In various instances, a match between the sample fingerprint profile and the sequencing fingerprint profiles in the truth fingerprint table may not be found because the sample fingerprint profile is that of a homozygous wild-type fingerprint profile. As such, no mutations are present in the targeted tandem repeat genomic region and the patient does not have the disease. The wild-type fingerprint profile may optionally be added to the truth fingerprint table.

### VII. Computing Environments: Machines, Software, and Interfaces

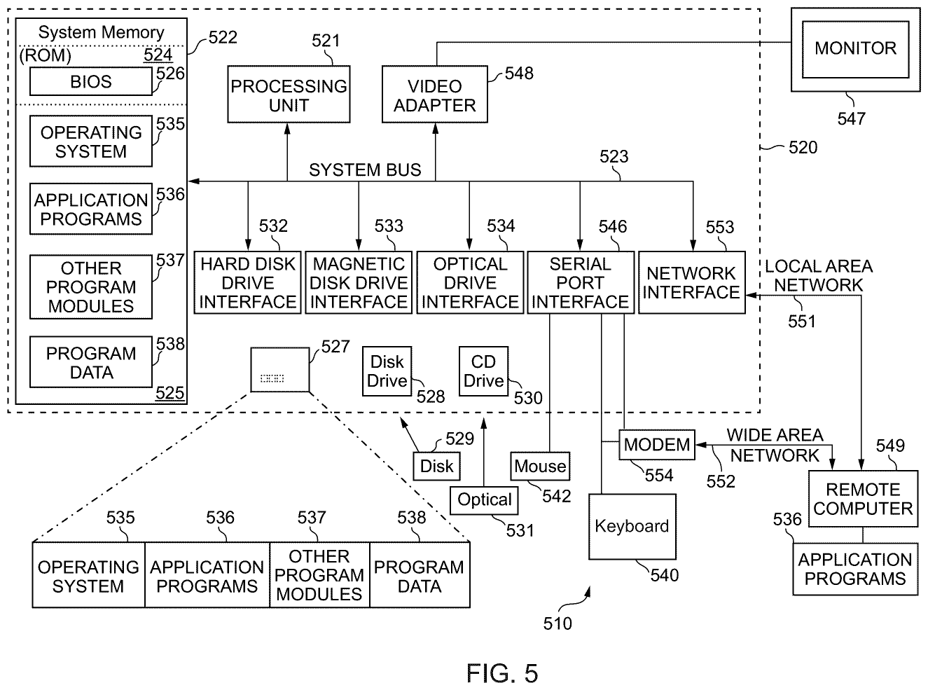

FIG. 5 illustrates a non-limiting example of a computing environment 510 in which various systems, methods, process, and data structures described herein may be implemented. The computing environment 510 is only one example of a suitable computing environment and is not intended to suggest any limitation as to the scope of use or functionality of the systems, methods, and data structures described herein. Neither computing environment 510 should be interpreted as having any dependency or requirement relating to any one or combination of components illustrated in computing environment 510. A subset of systems, methods, and data structures shown in FIG. 5 can be utilized in certain embodiments. Systems, methods, and data structures described herein are operational with numerous other general purpose or special purpose computing system environments or configurations. Examples of known computing systems, environments, and/or configurations that may be suitable include, but are not limited to, personal computers, server computers, thin clients, thick clients, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The computing environment 510 includes a computing device 520 (e.g., a computer or other type of machines such as sequencers, photocells, photo multiplier tubes, optical readers, sensors, etc.), including a processing unit 521, a system memory 522, and a system bus 523 that operatively couples various system components including the system memory 522 to the processing unit 521. There may be only one or there may be more than one processing unit 521, such that the processor of computing device 520 includes a single central-processing unit (CPU), or a plurality of processing units, commonly referred to as a parallel processing environment. The computing device 520 may be a conventional computer, a distributed computer, or any other type of computer.

The system bus 523 may be any of several types of bus structures including a memory bus or memory controller, a peripheral bus, and a local bus using any of a variety of bus architectures. The system memory may also be referred to as simply the memory and includes read only memory (ROM) 524 and random-access memory (RAM) 525. A basic input/output system (BIOS) 526, containing the basic routines that help to transfer information between elements within the computing device 520, such as during start-up, is stored in ROM 524. The computing device 520 may further include a hard disk drive 527 for reading from and writing to a hard disk, not shown, a magnetic disk drive 528 for reading from or writing to a removable magnetic disk 529, and an optical disk drive 530 for reading from or writing to a removable optical disk 531 such as a CD ROM or other optical media.

The hard disk drive 527, magnetic disk drive 528, and optical disk drive 530 are connected to the system bus 523 by a hard disk drive interface 532, a magnetic disk drive interface 533, and an optical disk drive interface 534, respectively. The drives and their associated computer-readable media provide non-volatile storage of computer-readable instructions, data structures, program modules and other data for the computing device 520. Any type of computer-readable media that can store data that is accessible by a computer, such as magnetic cassettes, flash memory cards, digital video disks, Bernoulli cartridges, random-access memories (RAMs), read only memories (ROMs), and the like, may be used in the operating environment.

A number of program modules may be stored on the hard disk 527, magnetic disk 528, optical disk 530, ROM 524, or RAM 525, including an operating system 535, one or more application programs 536, other program modules 537, and program data 538. A user may enter commands and information into the computing device 720 through input devices such as a keyboard 540 and pointing device (e.g., mouse) 542. Other input devices (not shown) may include a microphone, joystick, game pad, satellite dish, scanner, or the like. These and other input devices are often connected to the processing unit 521 through a serial port interface 546 that is coupled to the system bus 523, but may be connected by other interfaces, such as a parallel port, game port, or a universal serial bus (USB). A monitor 547 or other type of display device is also connected to the system bus 523 via an interface, such as a video adapter 548. In addition to the monitor 547, computers typically include other peripheral output devices (not shown), such as speakers and printers.

The computing device 520 may operate in a networked environment using logical connections to one or more remote computers, such as remote computer 549. These logical connections may be achieved by a communication device coupled to or a part of the computing device 520, or in other manners. The remote computer 549 may be another computer, a server, a router, a network PC, a client, a peer device, or other common network node, and typically includes many or all the elements described above relative to the computing device 520, although only memory storage devices has been illustrated in FIG. 5. The logical connections depicted in FIG. 5 include a local-area network (LAN) 551 and a wide-area network (WAN) 552. Such networking environments are commonplace in office networks, enterprise-wide computer networks, intranets, and the Internet, which all are types of networks.

When used in a LAN-networking environment, the computing device 520 is connected to the LAN 551 through a network interface or adapter 553, which is one type of communications device. When used in a WAN-networking environment, the computing device 520 often includes a modem 554, a type of communications device, or any other type of communications device for establishing communications over the WAN 552. The modem 554, which may be internal or external, is connected to the system bus 523 via the serial port interface 546. In a networked environment, program modules depicted relative to the computing device 520, or portions thereof, may be stored in the remote memory storage device. It is appreciated that the network connections shown are non-limiting examples and other communications devices for establishing a communications link between computers may be used.

### VIII. EXAMPLES

The models and techniques implemented in various embodiments may be better understood by referring to the following examples.

### Example 1: Materials and Methods

PCR amplification was performed using 0.25U AmpliTaq Gold DNA polymerase (ThermoFisher Scientific, Waltham, MA) in 1×PCR Buffer II, 1.5 mM MgCl2, 50 μM dNTPs, and 250 nM primers and the following cycling condition: 1 cycle at 95° C. for 10 minutes; 35 cycles of denaturation at 95° C. for 20 seconds, annealing at 60° C. for 30 seconds and extension at 72° C. for 60 seconds; 1 cycle at 72° C. for 3 minutes. Sanger sequencing was carried out using BigDye Terminator v3.1 Cycle Sequencing kit (ThermoFisher Scientific, Waltham, MA), and capillary electrophoresis on the ABI3730XL (ThermoFisher Scientific, Waltham, MA). SeqScape v2.5 (ThermoFisher Scientific, Waltham, MA) was used for sequence analysis and visualization.

### Example 2: CFTR polyTG/polyT Analysis of Simulated Samples

The CFTR polyTG/polyT tract variants are a unique case presenting challenges to an off-the-shelf NGS method. Specifically, the CFTR intronic region between exons 9 and 10 (on hg19/GRCh37) contains a string of TG-repeats, followed by a clinically significant polyT tract starting just six bases upstream of exon 10. The wild type takes the form of (TG)11(T)7, but shorter polyT tracts (5T or less) have been linked to reduced levels of CFTR mRNA and protein, which can potentially lead to CFTR-related disorders. However, since the lengths of polyTG and polyT tracts can vary between the two alleles, this region presents challenges in aligning and interpreting NGS data.

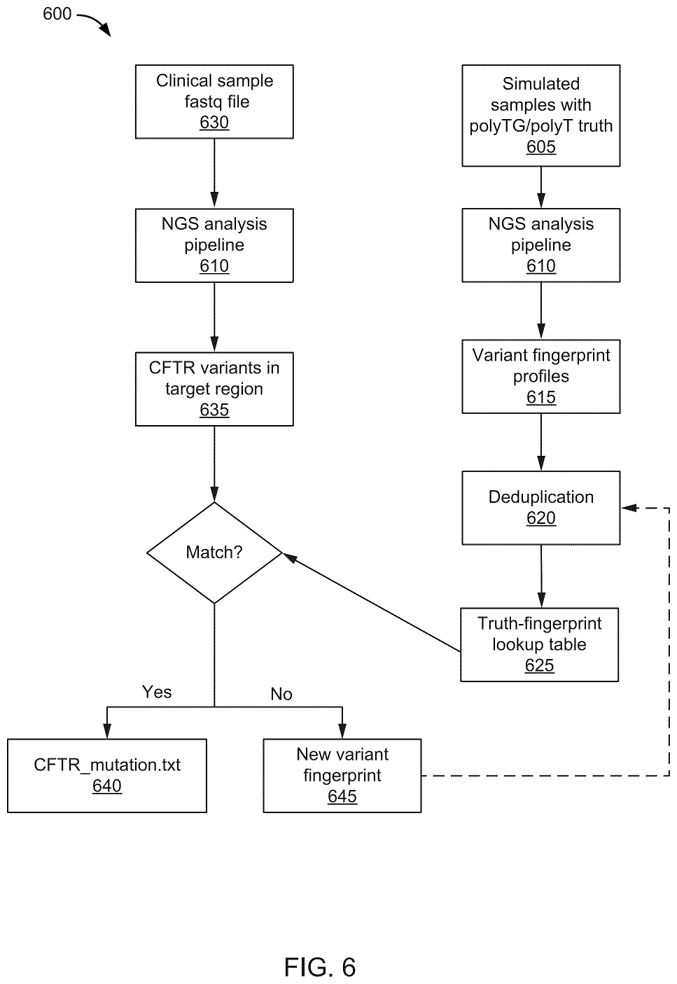

To address these challenges and others, a computational strategy was developed with a digital read-out for detecting NGS variants in the CFTR polyTG/polyT region (see FIG. 6). To address the computational burden, the NGS analysis pipeline was abbreviated to only consider ±500 bp on each side of polyTG polyT region at chr7:117188660-117188689 in hg19/GRCh37. In so doing, computational time was significantly reduced where multiple samples, e.g., over 200 samples, can be processed in less than a minute. As illustrated in FIG. 6, the computational strategy involves generating simulated samples that comprise all possible combinations of polyT and polyTG and analyzing the simulated samples in a bioinformatics pipeline to construct a comprehensive lookup table containing variant fingerprint profiles. The profiles were then used to match variant fingerprint profiles in clinical specimens to determine the lengths of polyT and polyTG in each sample. The accuracy of the approach was confirmed by Sanger sequencing, which is generally considered the gold standard for variant confirmation, but because Sanger utilizes an analog signal, it is time-consuming and challenging to disentangle two alleles with varying polyTG/polyT lengths. Application of this abbreviated NGS-based method to the analysis of polyTG/polyT regions in clinical samples not only offers an accurate and high-throughput solution, but also provides valuable insights into their patterns in the general population.

At box 605, a set of simulated variant samples comprising polyT and polyTG truths were generated to assess the impact of various polyT and polyTG repeat combinations on the performance of the updated NGS workflow. Instead of using the whole (FTR sequence to generate simulated samples, the region was shorted to include ±500 bp on each side of polyTG polyT region at chr7:117188660-117188689 in hg19/GRCh37. High quality polyT and polyTG simulated reads were generated using a synthetic next-generation sequencing reads tool (e.g., ART) with the following parameters: fragment length 300 bp, read length 150 bp, coverage 100×, with parameters for HiSeq 2500. In all, 13,689 simulated variant samples were generated from all combinations of TG:7-15 and T:1-12, were all generated samples had a predefined, known, number of Ts and TGs. Further, each simulated variant sample included two phased haplotypes, except cases where the two haplotypes were identical. For example, one haplotype could have linked (TG)12(T)5, where the other haplotype could be linked (TG)10(T)9.

At box 610, the simulated variant samples were processed by a NGS analysis pipeline. The NGS analysis pipeline outputs many variant tables (e.g., vcf, csv, and xml files). The features (e.g., chromosome number and region, variant type, reference sequence, variant alteration, etc.) from the variant tables are concatenated into one string, referred to as a mutation pattern, which is used as the variant fingerprint for the simulated polyT and polyTG variant sequences. To avoid inadvertently excluding the homopolymer polyT sequence, the homopolymer filter is disabled for the CFTR polyTG/polyT region from the NGS pipeline; however, variants in all other regions are filtered out if a variant is from a homopolymer region with length >10.

At box 615, the variant fingerprint profiles are reported. Table 2 shows an example of a simulated variant sample and its corresponding mutation pattern (e.g., fingerprint) identified by the NGS analysis pipeline.

TABLE 2

| Simulated Truth Sample | Fingerprint |
|---|---|
| TG_10_with_T_09 | 117188682_G_T_SNV_HET |
| TG_13_with_T_05 | 117188683_T_TGT_INS_HET |
| | 117188684_T_G_SNV_HET |

As variant fingerprints are reported, deduplication 620 is performed to remove any duplicated mutation patterns. Deduplication can be run as an inline process as the data is being written into the storage system (e.g., the truth-fingerprint lookup table 625) and/or as a background process to eliminate duplicates after the data is written to disk, significantly reducing storage capacity requirements. Examples of duplication events that could occur and be removed are described below.
a) When performing the simulation, some samples were simulated twice, for example sample (TG)12(T)5_(TG)10(T)9 is the same as sample (TG)10(T)9_(TG)12(T)5. If these cases reported the same mutation pattern, then the final lookup table will have only one entry with standardized naming convention.
b) Different simulated samples with different numbers of Ts and TGs (truths) outputting the same mutation pattern were also de-duplicated. For example, as illustrated in Table 3, two different simulated samples can report identical mutation patterns (e.g., fingerprints).

TABLE 3

| Simulated Truth Sample | Fingerprint |
|---|---|
| TG_11_with_T_06_TG_14_with_T_02 | 117188683_T_TG_INS_HET |
| | 117188685_T_G_SNV_HET |
| | 117188687_T_G_SNV_HET |
| | 117188688_TT_T_DEL_HET |
| TG_12_with_T_06_TG_13_with_T_02 | 117188683_T_TG_INS_HET |
| | 117188685_T_G_SNV_HET |
| | 117188687_T_G_SNV_HET |
| | 117188688_TT_T_DEL_HET |

Thus, samples with the same mutation pattern have concatenated name for the truth, e.g., “TG_11_with_T_06_TG_14_with_T_02; TG_12_with_T_06_TG_13_with_T_02”. The deduplicated mutation patterns and their paired simulated variant are used to construct the truth-fingerprint lookup table 625.

### Example 3: CFTR polyTG/polyT Analysis of Clinical Specimens

In addition to processing simulated variant samples (e.g., truth samples), the computational workflow depicted in FIG. 6 is also be used to process clinical samples. Clinical samples can be a cell-containing liquid or a tissue, for example a whole blood sample and/or a prenatal blood sample acquired by blood draw from a patient at risk of, diagnosed with, and/or being treated for cystic fibrosis. The clinical samples are processed for library construction and sequencing using any of the aforementioned methods. In some instances, the clinical sample is processed for targeted genotyping by sequencing using a bait-capture method. Baits are oligonucleotides designed to target specific RNA or genomic DNA fragments of interest through hybridization (e.g., ±500 bp on each side of polyTG polyT region at chr7:117188660-117188689 in hg19/GRCh37). Additionally or alternatively, the clinical sample is processed for WES. Following sequencing, a clinical sample FASTQ file 630 is output that includes the sequencing reads, and their associated quality scores.

At box 610, the clinical sample FASTQ file 630 was processed by the same NGS analysis pipeline 610 used for processing the simulated variant samples. The NGS analysis pipeline 610 detects polyTG/polyT variants located in the target region of CFTR and outputs tables (e.g., vcf, csv and xml file) containing the clinical sample variant and sample features (e.g., chromosome number and region, variant type, reference sequence, variant alteration, etc.). A filtering process can be used to remove any variants in regions outside of the target region. The features from the variant tables are concatenated into one string, referred to as a mutation pattern, which is used as the polyT and polyTG fingerprint for the clinical sample. To avoid inadvertently excluding the homopolymer polyT sequence, the homopolymer filter is removed from the NGS pipeline; however, variants in all other regions are filtered out if a variant is from a homopolymer region with length >10.

At box 635, the CFTR variant fingerprint profiles are reported and compared to the truth-fingerprint lookup table 625 to find the matching simulated polyT and polyTG lengths. If matching mutation patterns were found, the corresponding simulated sample would be reported into a CFTR_mutation.txt file 640. The CFTR_mutation.txt file 640 stores the final reported polyT/polyTG for the two haplotypes (from the two chromosome copies), plus the details of the input CFTR variants used to build the polyT/polyTG report. Table 4 shows an example of the CFTR variant details that can be included in the CFTR_mutation.text file 640.

TABLE 4

| | PolyT Alleles | | | | | | | | | |
|---|---|---|---|---|---|---|---|---|---|---|
| | Sample | | | | | | | | | |
| PolyTG | ID | Chr | Region | Ref | Alt | Type | AF | COV | CNT | ZYG |
| TG_11 T_05 | 36488190 | 7 | 117188688 | TTT | T | DEL | 44.16 | 77 | 34 | HET |
| TG_11 T_07 | | | | | | | | | | |

Abbreviations: Chr.: Chromosome; Ref: reference allele; Alt: alternative allele; Type: Variant type; AF: allele frequency; COV: total read coverage at the position specified by the region column; CNT: Variant read count at the position specified by the region column; ZYG: zygosity of the variant call at the position specified by the region column.

If a new mutation pattern (e.g., fingerprint) with a new sample truth label and/or an old mutation pattern with a new sample truth label were entered into the truth-fingerprint lookup table 625, these samples without a match were categorized as new variant fingerprint 645 and would repeat the deduplication 620 and match comparison with the truth-fingerprint lookup table 625 until a match was found.

In a small number of cases, two different sets of simulated samples reported the same set of variants. In all these cases, the polyT size was the same, but the TG repeat length was different based on simulated data.

### Example:

A. Variant set 1: TG_10_with_T_07_TG_10_with_T_09 (truth)
B. Variant set 2: TG_09_with_T_09_TG_11_with_T_07 (alternative explanation)

NGS read alignment can be used to resolve these ambiguous calls. In this example (FIG. 7B), NGS reads that mapped to the CFTR polyT/polyTG region were extracted for the sample and the strings from the polyTG/polyT stretch were compiled and counted. In so doing, the TG_10_with_T_07_TG_10_with_T_09 variant set was found to comprise the two dominant populations in this sample. Sanger sequencing was used to confirm a number of cases like this.

### Example 4: Sanger Confirmation

A proof-of-concept study was performed by using the NGS analysis pipeline to process 27 clinical samples with confirmed polyTG/polyT results from Sanger. As shown in Table 5, the NGS polyTG/polyT results were 100% concordant with the Sanger results.

TABLE 5

| | | | Agree |
|---|---|---|---|
| Sample ID | Haplotype 1 | Haplotype 2 | with Sanger |
| Sample 001 | TG[9]T[9] | TG[11]T[7] | Y |
| Sample 002 | TG[10]T[7] | TG[11]T[9] | Y |
| Sample 003 | TG[10]T[7] | TG[11]T[5] | Y |
| Sample 004 | TG[10]T[7] | TG[12]T[7] | Y |
| Sample 005 | TG[10]T[7] | TG[11]T[5] | Y |
| Sample 006 | TG[10]T[7] | TG[10]T[9] | Y |
| Sample 007 | TG[10]T[7] | TG[12]T[5] | Y |
| Sample 008 | TG[10]T[7] | TG[11]T[5] | Y |
| Sample 009 | TG[10]T[7] | TG[12]T[7] | Y |
| Sample 010 | TG[10]T[7] | TG[12]T[5] | Y |
| Sample 011 | TG[10]T[7] | TG[11]T[9] | Y |
| Sample 012 | TG[10]T[7] | TG[11]T[5] | Y |
| Sample 013 | TG[10]T[7] | TG[11]T[7] | Y |
| Sample 014 | TG[10]T[7] | TG[12]T[7] | Y |
| Sample 015 | TG[10]T[9] | TG[11]T[5] | Y |
| Sample 016 | TG[10]T[9] | TG[11]T[9] | Y |
| Sample 017 | TG[10]T[9] | TG[12]T[7] | Y |
| Sample 018 | TG[10]T[9] | TG[11]T[5] | Y |
| Sample 019 | TG[10]T[9] | TG[11]T[9] | Y |
| Sample 020 | TG[11]T[5] | TG[11]T[7] | Y |
| Sample 021 | TG[11]T[5] | TG[11]T[7] | Y |
| Sample 022 | TG[11]T[7] | TG[11]T[9] | Y |
| Sample 023 | TG[11]T[7] | TG[12]T[7] | Y |
| Sample 024 | TG[11]T[7] | TG[11]T[5] | Y |
| Sample 025 | TG[11]T[9] | TG[11]T[9] | Y |
| Sample 026 | TG[12]T[7] | TG[13]T[5] | Y |
| Sample 027 | TG[12]T[7] | TG[12]T[7] | Y |

FIG. 7 shows a comparison of Sanger sequencing results (FIG. 7A) compared to NGS sequencing results (FIG. 7B) for Sample 010. FIG. 7A shows chromatogram of the Sanger sequencing results for the CFTR polyTG/polyT region. A chromatogram represents the migration of labeled sequencing products via capillary electrophoresis. Fluorescence is detected at the end of the capillary, and signal intensity from four color channels, each representing a DNA base, is plotted on the y-axis relative to time on the x-axis. The topmost row (e.g., the multi-colored boxes) indicate the quality score of each assigned base. The quality score is the average quality value for all peaks in the trace with an assigned base. It serves as a metric for overall sequence quality. Generally, traces with quality score ≥40 are considered good quality. Peaks with quality scores <20 are likely to have a weak signal and/or high noise. Also provided is the original sequence of the polyTG/polyT region. FIG. 7B shows a bar graph illustrating that the polyT and polyTG NGS read count profile is consistent with the Sanger result. Overall, both methods detected Sample 010 has having a haplotype of TG[10]T[7]; TG[12]T[5], demonstrating that NGS computational process shown in FIG. 6 agrees with the gold standard Sanger method.

### IX. Additional Considerations

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes, but is not limited to, portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

What is claimed is:

1. A computer implemented method comprising:
generating, using a NGS simulation tool, sets of simulated variants and corresponding simulated sequencing read files for a targeted genomic region, wherein:
each of the sets of simulated variants from the sets of simulated variants comprise a first allele with zero or more mutations, and a second allele with zero or more mutations, and
the simulated sequencing read files comprise (i) a forward sequencing read file comprising at least 10,000 simulated reads, (ii) a reverse sequencing read file comprising at least 10,000 simulated reads, or both (i) and (ii);
detecting, using the simulated sequencing files, zero or more variations in the first allele, and zero or more variations in the second allele, wherein detecting comprises: inputting the simulated sequencing files into a sequencing analysis pipeline for alignment to a reference genome, identifying the zero or more variations between the first allele's simulated sequencing read file and the reference genome and the zero or more variations between the second allele's simulated sequencing read file and the reference genome, and outputting a variant table comprising the zero or more detected variants and their corresponding variant features;
concatenating the variant features for the zero or more detected variants in the first allele and the variant features for the zero or more detected variants in the second allele to generate a sequencing fingerprint profile; and
outputting, into a truth fingerprint table, the sets of simulated variants and their corresponding sequencing fingerprint profiles.

2. The computer implemented method of claim 1, wherein the targeted genomic region is ±500 base pairs of the CFTR polyTG/polyT region.

3. The computer implemented method of claim 1, wherein the first allele and the second allele comprise the same mutations, different mutations, or any combination thereof.

4. The computer implemented method of claim 1, wherein the sequencing fingerprint profile comprises the starting coordinate for the variant, the reference genome nucleic acid base, the alternative nucleic acid base, the variant type, the zygosity of the variant, or any combination thereof.

5. The computer implemented method of claim 1, wherein outputting further comprising deduplicating the sequencing fingerprint profile to remove simulated variants from the truth fingerprint table that generate the same sequencing fingerprint profile.

6. A computer implemented method comprising:
performing a next generation sequencing assay on a sample, wherein the NGS assay generates sample sequencing read files for the targeted genomic region, wherein the sample sequencing read files comprise (i) a forward sequencing read file, (ii) a reverse sequencing read file, or both (i) and (ii);
processing, using a sequencing analysis pipeline, the sample sequencing read files for variant detection, wherein processing comprises: inputting the sample sequencing read files into a sequencing analysis pipeline for alignment to a reference genome, identifying zero or more variations between the sequencing read files and the reference genome, and outputting a variant table comprising the zero or more detected variants and their corresponding variant features;
concatenating the variant features for the zero or more detected variants to generate a sample fingerprint profile;
accessing a truth fingerprint table comprising sets of simulated variants and their corresponding sequencing fingerprint profiles;
cross-referencing the sample fingerprint profile with the sequencing fingerprint profiles in the truth fingerprint table to determine whether a match exists;
when a match exists between the sample fingerprint profile and the sequencing fingerprint profiles in the truth fingerprint table, (i) generating a report comprising the sample fingerprint profile and the detected variants in the sample; and
when a match does not exist between the sample fingerprint profile and the sequencing fingerprint profiles in the truth fingerprint table, (i) updating the truth fingerprint table to include the sample fingerprint profile, (ii) repeating the cross-referencing for the sample fingerprint table, and (iii) generating a report comprising the sample fingerprint profile and the detected variants in the sample.

7. The computer implemented method of claim 6, wherein the sample is a cell-containing liquid or a tissue comprising nucleic acid molecules.

8. The computer implemented method of claim 7, wherein the sample is a blood sample.

9. The computer implemented method of claim 6, wherein the NGS assay is a targeted NGS assay for a targeted genomic region.

10. The computer implemented method of claim 9, wherein the targeted genomic region is ±500 base pairs of the CFTR polyTG/polyT region.

11. The computer implemented method of claim 6, further comprising:
when the sequencing analysis pipeline is unable to make a definitive variant call from the sample sequencing read files:
accessing, using the sequencing analysis pipeline, the sample sequencing read files to count the number of reads that align to the detected variant; and
resolving, based on the sequencing read counts, the fingerprint profile for the detected variant.

12. A system comprising:
one or more processors; and
one or more computer-readable media storing instructions which, when executed by the one or more processors, cause the system to perform operations comprising:
generating, using a NGS simulation tool, at least 5,000 sets of simulated variants and corresponding simulated sequencing read files for a targeted tandem repeat genomic region, wherein:
each of the sets of simulated variants from the at least 5,000 sets of simulated variants comprise a first allele with zero or more mutations, and a second allele with zero or more mutations, and
the simulated sequencing read files comprise (i) a forward sequencing read file comprising at least 10,000 simulated reads, (ii) a reverse sequencing read file comprising at least 10,000 simulated reads, or both (i) and (ii);
detecting, using the simulated sequencing files, zero or more variations in the first allele and zero or more variations in the second allele, wherein detecting comprises: inputting the simulated sequencing files into a sequencing analysis pipeline for alignment to a reference genome, identifying the zero or more variations between the first allele's simulated sequencing read file and the reference genome and the zero or more variations between the second allele's simulated sequencing read file and the reference genome, and outputting a variant table comprising the zero or more detected variants and their corresponding variant features;
concatenating the variant features for the zero or more detected variants in the first allele and the variant features for the zero or more detected variants in the second allele to generate a sequencing fingerprint profile;
outputting, into a truth fingerprint table, the at least 5,000 sets of simulated variants and their corresponding sequencing fingerprint profiles;
performing an NGS assay on a sample, wherein the NGS assay generates sample sequencing read files for the targeted tandem repeat genomic region, wherein the sample sequencing read files comprise (i) a forward sequencing read file, (ii) a reverse sequencing read file, or both (i) and (ii);
processing, using a sequencing analysis pipeline, the sample sequencing read files for variant detection, wherein processing comprises: inputting the sample sequencing read files into a sequencing analysis pipeline for alignment to a reference genome, identifying zero or more variations between the sample sequencing read files and the reference genome, and outputting a variant table comprising the zero or more detected variants and their corresponding variant features;
concatenating the variant features for the zero or more detected variants to generate a sample fingerprint profile;
cross-referencing the sample fingerprint profile with the sequencing fingerprint profiles in the truth fingerprint table to determine whether a match exists;
when a match exists between the sample fingerprint profile and the sequencing fingerprint profiles in the truth fingerprint table, (i) generating a report comprising the sample fingerprint profile and the detected variants in the sample; and
when a match does not exist between the sample fingerprint profile and the sequencing fingerprint profiles in the truth fingerprint table, (i) updating the truth fingerprint table to include the sample fingerprint profile, (ii) repeating the cross-referencing for the sample fingerprint table, and (iii) generating a report comprising the sample fingerprint profile and the detected variants in the sample.

13. The system of claim 12, wherein the tandem repeat genomic region is ±500 base pairs of the CFTR polyTG/polyT region.

14. The system of claim 12, wherein the first allele and the second allele comprise the same mutations, different mutations, or any combination thereof.

15. The system of claim 12, wherein the sequencing fingerprint profile comprises the starting coordinate for the variant, the reference genome nucleic acid base, the alternative nucleic acid base, the variant type, the zygosity of the variant, or any combination thereof.

16. The system of claim 12, wherein outputting further comprising deduplicating the sequencing fingerprint profile to remove simulated variants from the truth fingerprint table that generate the same sequencing fingerprint profile.

17. The system of claim 12, wherein the sample is a cell-containing liquid or a tissue comprising nucleic acid molecules, and wherein the sample is a blood sample.

18. The system of claim 10, wherein the NGS assay is a targeted NGS assay for a tandem repeat genomic region, and wherein the tandem repeat genomic region is ±500 base pairs of the CFTR polyTG/polyT region.

19. The system of claim 10, wherein the sample fingerprint profile comprises the starting coordinate for the variant, the reference genome nucleic acid base, the alternative nucleic acid base, the variant type, the zygosity of the variant, or any combination thereof.

20. The system of claim 10, further comprising:
when the sequencing analysis pipeline is unable to make a definitive variant call from the sample sequencing read files:
accessing, using the sequencing analysis pipeline, the sample sequencing read files to count the number of reads that align to the detected variant; and
resolving, based on the sequencing read counts, the fingerprint profile for the detected variant.
